(19) United States
(12) Patent Application Publication
(10) Pub. No.: US 2017/0211120 A1
(43) Pub. Date: Jul. 27, 2017

(54) Pathogen Identification in Complex Biological Fluids

(71) Applicants: Robert K. Ernst, Silver Spring, MD (US); David Goodlett, Baltimore, MD (US); Lisa Leung, Laurel, MD (US)

(72) Inventors: Robert K. Ernst, Silver Spring, MD (US); David Goodlett, Baltimore, MD (US); Lisa Leung, Laurel, MD (US)

(73) Assignee: University of Maryland, Baltimore, Baltimore, MD (US)

(21) Appl. No.: 15/410,948

(22) Filed: Jan. 20, 2017

Related U.S. Application Data
(60) Provisional application No. 62/281,523, filed on Jan. 21, 2016.

(51) Int. Cl. C12Q 1/04; H01J 49/00; G01N 33/92; G01N 27/62; C12Q 1/18; G01N 33/49

(52) U.S. Cl. CPC C12Q 1/04; C12Q 1/18; G01N 33/49; G01N 33/92; G01N 27/622; H01J 49/004; H01J 49/0036

(57) ABSTRACT

Provided herein are methods for rapidly identifying a microbe, such as a pathogen, from a biological sample including, blood, urine, wound effluent, stool, serum, and bronchoalveolar lavage fluid. The method comprises obtaining the sample from the subject and performing a spectrometric analysis of the lipids in the microbe to obtain a profile. The profile obtained is compared with a molecular mass lipid profile of known microbes for identification. Also provided is a method for identifying one or more antimicrobial drugs effective to treat a microbial strain in a subject.

### CROSS-REFERENCE TO RELATED APPLICATIONS

This non-provisional application claims benefit of priority under 35 U.S.C. §119(e) of provisional application U.S. Ser. No. 62/281,523, filed Jan. 21, 2016, the entirety of which is hereby incorporated by reference.

### FEDERAL FUNDING LEGEND

This invention was made with government support under Grant Number GM111066 awarded by the National Institutes of Health. The government has certain rights in the invention.

### BACKGROUND OF THE INVENTION

Field of the Invention

The invention relates generally to the field of medicine and clinical microbiology. In particular, the invention relates to a diagnostic method for rapid identification of pathogenic species from complex biological fluids.

Description of the Related Art

Clinicians need to rapidly and accurately identify pathogens during life-threatening infections. Current methods require culturing microorganisms, such as bacteria on solid medium to obtain a pure colony and usually requires multiple rounds of replication to permit diagnosis, which can often require significant time. However, there are critical failings due to the inability to differentiate closely related species or multiple organisms from complex biological fluids. There is clearly a need for a rapid method which allows for rapid assay and identification of microorganisms, such as bacteria and fungi, from complex fluids without the need to grow the organisms out on differential and complex media, a process often requiring several days before identification of the microorganism species can be accurately ascertained.

It would be beneficial, therefore, to find a method that allows for rapid (within hours) and accurate identification of a variety of microorganisms such as Gram-positive and Gram-negative bacteria and fungi, from clinically relevant samples by mass spectrometry for lipid analysis of the different pathogen types. The prior art is deficient in this respect. The present invention fulfills this longstanding need and desire in the art.

### SUMMARY OF THE INVENTION

The present invention is directed to a method for rapidly identifying a microbe. This method comprises obtaining a biological sample from a subject and determining, via spectrometry, a molecular mass profile of microbial lipids either extracted from the microbe or from microbial cells. The molecular mass profile of the lipids from the microbe is compared with the molecular mass profile of lipids from a known microbe. An identical profile indicates the identity of the microbe in the biological sample. The present invention is directed to a related method that further comprises isolating the microbe from the biological sample.

The present invention also is directed to a method for rapidly identifying a pathogenic bacterium in a blood sample. This method comprises obtaining the blood sample from a subject and extracting lipids from the bacterial pathogen at zero passage. The molecular mass profile of the extracted lipids is determined via spectrometry. The molecular mass profile of the extracted lipids is compared with the molecular mass profile of lipids from a known pathogenic bacterium. An identical profile indicates the identity of the pathogenic bacterium. The present invention is directed to a related method that further comprises isolating the pathogenic bacterium from the biological sample.

The present invention is directed further to a method for identifying one or more antimicrobial drugs effective to treat a microbial strain in a subject in need of such treatment. This method comprises obtaining a blood sample from the subject and extracting lipids from microbes in the sample. A spectrographic analysis of the extracted lipids is performed to obtain a molecular mass profile thereof. The extracted lipids profile are compared with a library of known lipid profiles of strains of pathogenic microbes to identify the strain of pathogenic microbe in the biological sample followed by identifying one or more antimicrobial drugs effective to treat the identified microbial strain.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 illustrates the strategy of mass spectrometric analysis of Escherichia coli lipid A from blood bottles.

FIGS. 2A-2H shows E. coli inoculated into blood bottles detected by MALDI-TOF analysis of lipid A. FIG. 2A shows E.coli W3110 grown in nutrient rich medium. FIG. 2B shows E.coli W3110 grown in a blood bottle for 24 hours at 37° C. FIG. 2C shows E. coli W3110 grown in a blood bottle for 2 hours at 37° C. and sampled with differential centrifugation. FIG. 2D shows E. coli W3110 inoculated at 10⁸ CFU/mL in a blood bottle and grown for 4 hours at 37° C. FIG. 2E shows E. coli W3110 inoculated at 10⁶ CFU/mL in a blood bottle and grown for 6 hours at 37° C. FIG. 2F shows E. coli W3110 inoculated at 10⁶ CFU/mL in a blood bottle and grown for 24 hours at 37° C. FIG. 2G shows E. coli W3110 inoculated at 10⁸ CFU/mL in an aerobic blood bottle with neutralization resin. FIG. 2H shows E. coli W3110 inoculated at 108 CFU/mL in pediatric blood bottle with neutralization resin.

FIG. 3 shows the mass spectrometric analysis of pathogenic species of Pseudomonas aeruginosa PAO1 done in O+ blood sample.

FIGS. 4A-4B shows the mass spectrometric analysis of Acinetobacter baumannii done in blood sample. FIG. 4A shows the mass spectrometric analysis of Acinetobacter baumannii in standard aerobic bottles ˜10¹ intial inoculum at t₆. FIG. 4B shows the mass spectrometric analysis of Acinetobacter baumannii in standard aerobic bottles ˜10⁶ intial inoculum cells at t₆

FIGS. 5A-5D shows the mass spectrometric analysis of Staphylococcus aureus done in blood sample. FIG. 5A shows the mass spectrometric analysis of Staphylococcus aureus MRSA M2 in standard aerobic bottle at ˜10¹ intial inoculum at 24 hours (t₂₄). FIG. 5B shows the mass spectrometric analysis of Staphylococcus aureus MRSA M2 in standard aerobic bottle at ˜10⁷ intial inoculum at t₂₄. FIG. 5C shows the mass spectrometric analysis of Staphylococcus aureus MRSA M2 in standard anaerobic bottle at ˜10⁷ intial inoculum at t₂₄. FIG. 5D shows the mass spectrometric analysis of Staphylococcus aureus MRSA NRS123 in standard anaerobic bottle at ˜10⁸ intial inoculum at t₂₄.

FIG. 6 shows the mass spectrometric analysis of Klebsiella pneumoniae B6 in standard aerobic bottle at ˜10⁸ intial inoculum at t₆.

FIGS. 7A-7R show the mass spectrometric analysis of pathogenic species done in urine. FIG. 7A shows the mass spectrometric analysis of Arthrobacter pigmenti. FIG. 7B shows the mass spectrometric analysis of Bacillus cereus. FIG. 7C shows the mass spectrometric analysis of Bacillus pumilus. FIG. 7D shows the mass spectrometric analysis of Brevundimonas diminuta. FIG. 7E shows the mass spectrometric analysis of Candida albicans. FIG. 7F shows the mass spectrometric analysis of Enterococcus faecalis. FIG. 7G shows the mass spectrometric analysis of Exiguobacterium. FIG. 7H shows the mass spectrometric analysis of Micrococcus luteus. FIG. 7I shows the mass spectrometric analysis of Moraxella osloensis. FIG. 7J shows the mass spectrometric analysis of Paenibacillus lautus. FIG. 7K shows the mass spectrometric analysis of Pseudomonas oryzihabitans. FIG. 7L shows the mass spectrometric analysis of Pseudomonas stutzeri. FIG. 7M shows the mass spectrometric analysis of Rhodococcus opacus. FIG. 7N shows the mass spectrometric analysis of Roseomonas mucosa. FIG. 7O shows the mass spectrometric analysis of Rothia amarae. FIG. 7P shows the mass spectrometric analysis of Staphylococcus aureus. FIG. 7Q shows the mass spectrometric analysis of Staphylococcus capitis. FIG. 7R shows the mass spectrometric analysis of Staphylococcus cohnii.

FIG. 8 shows the mass spectrometric analysis of one E.coli fecal pellet incubated overnight in liquid medium.

FIG. 9 shows the mass spectrometric analysis of Francisella species done in rich medium, wound effluent, bronchoalveolar lavage fluid and serum.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the terms “microbe” and “microorgansim” are interchangeable and includes pathogenic, non-pathogenic and commensal organisms, such as, but not limited to, bacteria, viruses, protozoas, and fungi.

As used herein, the phrase “zero passage” refers to the culture before medium replacement. The cells are grown for a period of time in one dish. When the cells are transferred to a second dish the cells are considered to be passaged. The first plating of cells is considered to be zero passage. For the purposes of this invention, “zero passage” also refers to extraction of lipids from microbes or analyzing lipids from whole microbial cells comprising a sample, particularly a blood sample, without first culturing the microbes for any period of time.

In one embodiment of the invention, there is provided a method for rapidly identifying a microbe, comprising obtaining a biological sample from a subject; determining, via spectrometry, a molecular mass profile of microbial lipids; and comparing the molecular mass profile of the lipids from the microbe with a molecular mass profile of lipids from a known microbe wherein an identical profile indicates the identity of the microbe in the biological sample.

Further to this embodiment the method comprises isolating the microbe from the biological sample. In an aspect of both embodiments the determining step may comprise extracting lipids from the microbe prior to the spectrometry; or performing spectrometry on microbial cells. The extracting step may comprise hydrolyzing the pathogenic cells by heat assisted mild acid hydrolysis.

Also in both embodiments the spectrometry may be mass spectrometry, tandem mass spectrometry (MS/MS) including multiple reaction monitoring and linked scans or ion mobility spectrometry (IMS). Representative types of mass spectrometry include, but are not limited to, matrix-assisted laser desorption/ionization-time-of-flight mass spectrometer (MALDI-TOF MS), Fourier transform ion cyclotron resonance mass spectrometry (FTICR-MS), ion trap, quadrupole, magnetic sector, Q-TOF, or triple quadrupole, platforms, tandem MS, infusion-based electro spray ionization (ESI) coupled to ion trap tandem mass spectrometry (ITMSⁿ), surface acoustic wave nebulization (SAWN) technology, including SAWN on any mass analyzer (e.g. quadrupole TOF-MS (QTOF) or SAWN-ion trap (IT) MS).

In addition, the lipid may be lipid A, Lipoteichoic Acid, a glycolipid, or cardiolipin. Furthermore, the microbe may be a pathogen, a non-pathogen or a commensal bacterium. Representative types of pathogens which may be detected using this method include but are not limited to Acinetobacter, Actinomyces, Arthrobacter, Burkholderia, Bacillus, Bacteroides, Bordetella, Borrelia, Brevundimonas, Brucella, Candida, Clostridium, Corynebacterium, Campylobacter, Deinococcus, Escherichia, Enterobacter, Enterococcus, Erwinia, Eubacterium, Exiguobacterium, Flavobacterium Francisella, Gluconobacter, Helicobacter, Intrasporangium, Janthinobacterium, Klebsiella, Kingella, Legionella, Leptospira, Mycobacterium, Moraxella, Micrococcus, Neisseria, Oscillospira, Proteus, Pseudomonas, Providencia, Paenibacillus, Rickettsia, Rhodococcus, Roseomonas, Rothia, Salmonella, Serratia, Staphylococcus, Shigella, Spinillum, Streptococcus, Stenotrophomonas Treponema, Ureaplasma, Vibrio, Wolinella, Wolbachia, Xanthomonas, Yersinia, and Zoogloea.

In a non-limiting aspect of these embodiments, the microbe is a Gram-negative bacterium and the lipid is lipid A, a glycolipid or cardiolipin. In another non-limiting aspect the microbe is a Gram-positive bacterium and the lipid is Lipoteichoic Acid, a glycolipid or cardiolipin. In yet another non-limiting aspect, the microbe may be a fungus and the lipid may be a glycolipid or cardiolipin or other fungal lipid. In all embodiments and aspects thereof representative biological samples include, but are not limited to, blood, urine, stool, serum, wound effluent or bronchoalveolar lavage fluid.

In another embodiment of the invention, there is provided a method for rapidly identifying a pathogenic bacterium in a blood sample comprising obtaining the blood sample from a subject; extracting lipids from the bacterial pathogen at zero passage; determining, via spectrometry, a molecular mass profile of the extracted lipids; and comparing the molecular mass profile of the extracted lipids with a molecular mass profile of lipids from a known pathogenic bacterium wherein an identical profile indicates the identity of the pathogenic bacteria.

Further to this embodiment the method comprises isolating the pathogenic bacterium from the blood sample. In this further embodiment, the isolating step may comprise separating the pathogenic bacterial cells from human cells via a low speed centrifugation. In both embodiments the extracting step may comprise hydrolyzing the pathogenic cells by a heat assisted mild acid hydrolysis.

In both embodiments the spectrometry may be mass spectrometry, a tandem mass spectrometry or ion mobility spectrometry. Particular types of spectrometry are as described supra.

In one aspect, the pathogenic bacterium is a Gram-negative bacterium and the lipid may be lipid A, a glycolipid or cardiolipin. In another aspect, the pathogen is a

Gram-positive bacterium and the lipid may be Lipoteichoic Acid, a glycolipid or cardiolipin. Representative types of pathogens which may be detected using this method include, but are not limited to, Acinetobacter, Actinomyces, Arthrobacter, Burkholderia, Bacillus, Bacteroides, Bordetella, Borrelia, Brevundimonas, Brucella, Candida, Clostridium, Corynebacterium, Campylabacter, Deinococcus, Escherichia, Enterobacter, Enterococcus, Erwinia, Eubacterium, Exiguobacterium, Flavobacterium, Francisella, Gluconobacter, Helicobacter, Intrasporangium, Janthinobacterium, Klebsiella, Kingella, Legionella, Leptospira, Mycobacterium, Moraxella, Micrococcus, Neisseria, Oscillospira, Proteus, Pseudomonas, Providencia, Paenibacillus, Rickettsia, Rhodococcus, Roseomonas, Rothia, Salmonella, Serratia, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

In yet another embodiment of the invention, there is provided a method for identifying one or more antimicrobial drugs effective to treat a microbial strain in a subject in need of such treatment, comprising obtaining a biological sample from the subject, extracting lipids from the pathogenic microbe comprising the sample, performing a spectrographic analysis of the extracted lipids to obtain a molecular mass profile thereof, comparing the extracted lipids profile with a library of known lipid profiles of strains of pathogenic microbes to identify the strain of pathogenic microbe in the biological sample and identifying one or more antimicrobial drugs effective to treat the identified microbial strain.

In this embodiment the performing step comprises analysis via mass spectrometry, a tandem mass spectrometry or ion mobility spectrometry as described. Representative types of mass spectrometry are as described supra. Also in the method described supra the spectrographic analysis of lipids differentiates among pathogenic microbes at the genus, species, sub-species or strain level.

In this embodiment, the pathogenic microbe is a Gram-negative bacterium, a Gram-positive bacterium or a fungus. Also in aspects of this embodiment, the Gram-negative bacterial lipid may be lipid A, a glycolipid or cardiolipin, the Gram-positive bacterial lipid may be Lipoteichoic Acid, a glycolipid or cardiolipin and the fungal lipid may be a glycolipid precursor of one or both of lipid A or Lipoteichoic Acid or a cardiolipin. Representative biological samples include, but are not limited to, blood, urine, stool, serum, wound effluent, or bronchoalveolar lavage fluid.

Provided herein are methods for rapidly identifying a variety of microbes from biological samples. This method has significant utility for accurately and rapidly identifying closely related pathogens during life threatening infections which circumvents the need to culture an organism to obtain sufficient amounts for analysis and/or testing or to ensure purity. The methods described herein produce an identification within hours rather than days, allowing for better antibiotic and antifungal stewardship. Moreover, these methods are useful to obtain information on antibiotic resistance markers depending on the pathogenic background, which can be used to inform therapeutic treatment.

Generally, the method comprises obtaining a biological sample from a subject and determining via spectrometry or performing a spectrometric analysis of the microbial lipids. Optionally, the microbe may be isolated from the sample or, alternatively, the whole microbial cell may be analyzed. For example, lipid analysis may utilize, but is not limited to, mass spectroscopic methods detailed and described in U.S. Pat. No. 9,273,339, the entirety of which is hereby incorporated by reference. The microbe in the sample is identified by comparing the molecular mass spectrometric profile of the microbial lipid(s) with a library of known microbial mass spectrometric profiles such as those described in U.S. Pat. No. 9,273,339.

Spectrometric analysis may be performed on lipids extracted and isolated from or on whole cells of Gram-negative bacteria, Gram-positive bacteria, either aerobic or anaerobic, and fungi, particularly fungal pathogens, with discrimination among strains with a high degree of identify or similarity, such as those identified in Table 1. Particularly, mass spectrometric analysis may be performed on lipids of one or more pathogens in a blood sample with zero passage culturing. The method is accurate for detecting as few as 10¹-10⁹ pathogenic cells present in the sample. Non-limiting examples of microbial lipids are lipid A, Lipoteichoic acid, a glycolipid, such as, but not limited to, glycolipid precursors of the lipid A and/or Lipoteichoic acid, a cardiolipin, glycolipids, cardiolipin, or sphingolipids, glycerolipids, glycerophospholipids, sterol lipids, prenol lipids, polyketides, etc. or combinations thereof.

As described herein, generally spectrometric methods may comprise mass spectrometry, a tandem mass spectrometry (MS/MS) including multiple reaction monitoring and linked scans or ion mobility spectrometry as are well known in the art. Particularly, ion mobility spectrometry is useful for multiple reaction monitoring. As a type or subset of tandem mass spectrometry, IMS enables organism specific mass channel assays where all m/z channels are no longer scanned. Only those channels where the organisms specific signature ions are expected to be found are scanned. Alternatively, linked scans, another type or subset of tandem mass spectrometry, enables a specific analysis for only certain kinds of lipids by observing their functional groups by tandem MS.

Also provided is a method for identifying one or more antimicrobial drugs effective to treat a microbial strain. The methods described herein enable a high level of discrimination among similar and related pathogens. One particular strain can be identified in a patient sample thereby enabling a treatment protocol to be planned for the subject best suited to treat the identified pathogenic microbe. This is particularly useful against antibiotic-resistant strains, such as methicillin resistant Staphylococcus aureus and acquired polymyxin resistant Klebsiella spp., Pseudomonas spp. and Acinetobacter spp. or naturally resistant Proteus spp., Serratia spp. and Burkholderia spp. Because the results are rapidly obtained without having to culture the sample, a subject can be monitored for acquisition of opportunistic pathogens.

The following examples are given for the purpose of illustrating various embodiments of the invention and are not meant to limit the present invention in any fashion. Embodiments of the present invention are better illustrated with reference to the Figure(s), however, such reference is not meant to limit the present invention in any fashion.

### EXAMPLE 1

### Blood Culture Growth of Microorganisms and Micro-extraction of Lipid A, Lipoteichoic Acid, Glycolipids and/or Cardiolipin for Mass Spectrometry Analysis

Sterile blood is obtained from the University of Maryland Medical Center (UMMC) blood bank and is stored at 4° C. upon receipt. Commercial blood culture bottles are available for aerobic, anaerobic or slow-growing organisms or contain specialized additives for antibiotic neutralization or blood cell lysis. For purposes of performing the method, different blood cultures bottles can be used including, but not limited to, bottles manufactured by Becton Dickinson and Company (BD) and BioMerieux. Moreover, any of a variety of culture media used to support the growth of Gram-positive and Gram-negative bacteria, including aerobes and anaerobes, can be utilized in this method.

### Preparation of 1M Ammonium Hydroxide Solution

Add 3.89 mL of 0.9 g/mL ammonium hydroxide to 96.1 mL endotoxin free H₂O for 100 mL of 1M Ammonium hydroxide.

### Preparation of Blood Culture (Day 1)

Remove the flip-off cap from the blood culture (BC) bottle and swab the septum with alcohol. Use a 10 mL syringe with a 26G blunt tip needle to draw 5-10 mL blood from blood bag. Invert syringe and switch needle for Vacutainer holder to transfer blood to blood culture bottle. The vacuum of the bottle should pull the blood in without having to depress the plunger; this ensures that the bottle hasn't been compromised. Use a 1 mL syringe to transfer bacterial inoculums into blood culture bottle either directly from liquid culture grown overnight or diluted appropriately in rich media. Grow at 37° C. with shaking immediately after inoculation.

### Sampling and Processing of Blood Culture Bottle (Day 1)

Use a 1-5 mL syringe to remove 1-2 mL culture at set time points. Swab septum with alcohol each time. Transfer it to 2 mL sterile microcentrifuge tube and spin tubes at 1100 rpm for 10 minutes. This pellets the human blood cells. Optionally transfer a small volume to a 96-well tissue culture plate to prepare serial dilutions in phosphate buffer saline (PBS) and to plate in triplicate on a lysogenic broth (LB) agar plate for enumeration. Transfer the supernatant to a 1.7 mL screw-cap tube and spin tube at 4000 rpm for 10 minutes. This pellets bacterial cells. Discard supernatant. Bacterial pellets are frozen at −20° C. or extracted immediately.

### Ammonium Isobutyrate Extraction of Glycolipids (Day 1)

Thaw the pellet and resuspend the wet pellet with 400 μL of a 5:3, v/v, 70% isobutyric acid:1M ammonium hydroxide solution (250 μL of isobutyric acid+150 μL Ammonium hydroxide). Incubate at 100° C. for 30-45 minutes with vortexing every ˜15 min (performed in fume hood). Cool the tube on ice and centrifuge at 2,000×g for 15 min. Remove the supernatant to a new tube with 400 μL endotoxin free water (1:1 v/v), put on the surrogate cap with a hole poked into the top to allow sublimation, then freeze and lyophilize overnight. Lyophilization generates a fluffy, white pellet (1).

### Processing of Extracts and MS Analysis (Day 2)

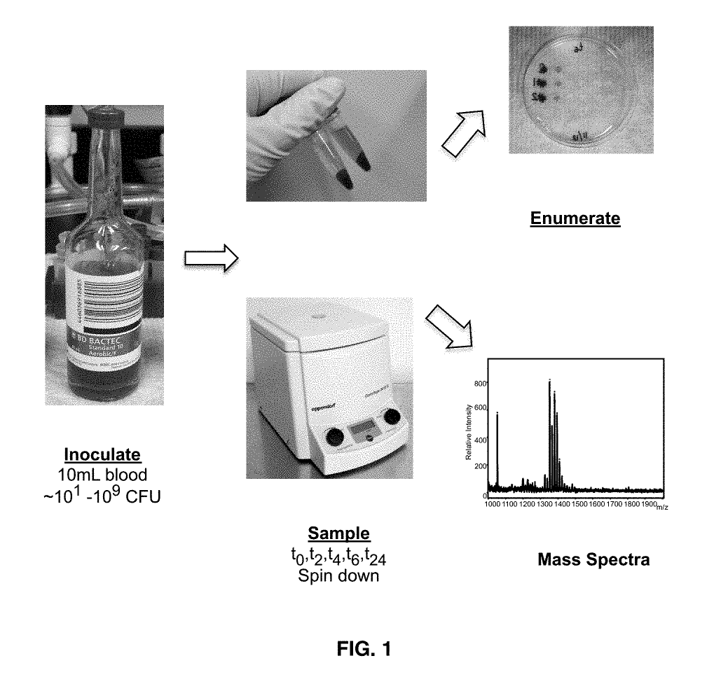

Wash sample with 1 mL of methanol, sonicate to resuspend and disrupt micelles, 5 minutes or more. Centrifuge at 10,000×g for 5 minutes, carefully aspirate methanol. Wash again with methanol. Solubilize and extract Lipid A in 25-190 μL of 3:1.5:0.25, v/v/v, chloroform:methanol:water, i.e. 120/60/10 μL. Vortex well. Optionally, add about 5 grains of Dowex 50WX8 (H+) and vortex well to desalt (e.g. Ca2+, Mg2+, and Na+). This enhances negative mode MALDI-MS sensitivity. Vortex and centrifuge at 8,000×g for 5 minutes. Spot 1 μL onto the MALDI target plate, followed by 1 μL of fresh matrix, for example, 10 [mg/mL] norharmane in 2:1, v/v, chloroform:methanol). Mass spectra are recorded in negative ion mode at 60-70% laser intensity and 900 laser shots per spectrum using a Bruker Microflex LRF MALDI-TOF (2) (FIGS. 1-6).

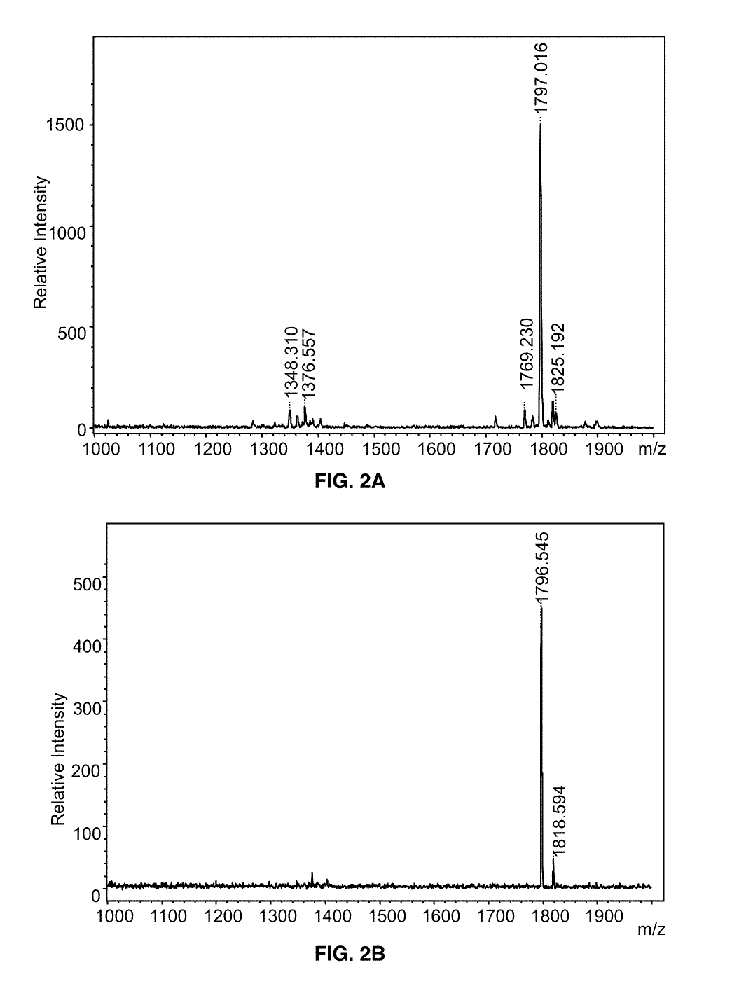

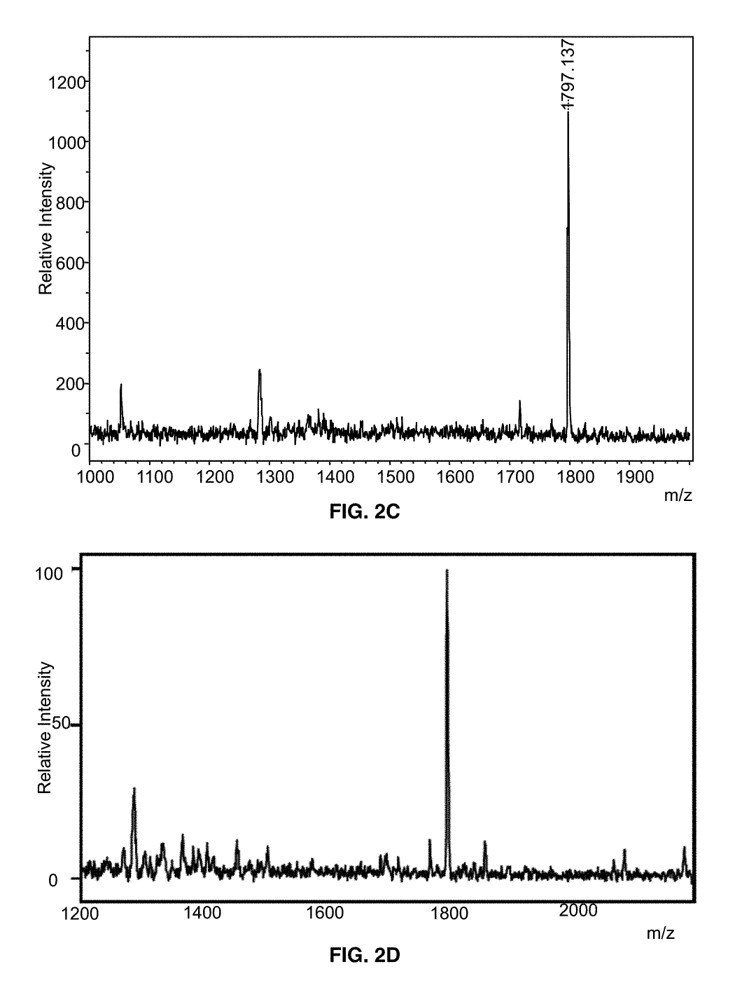

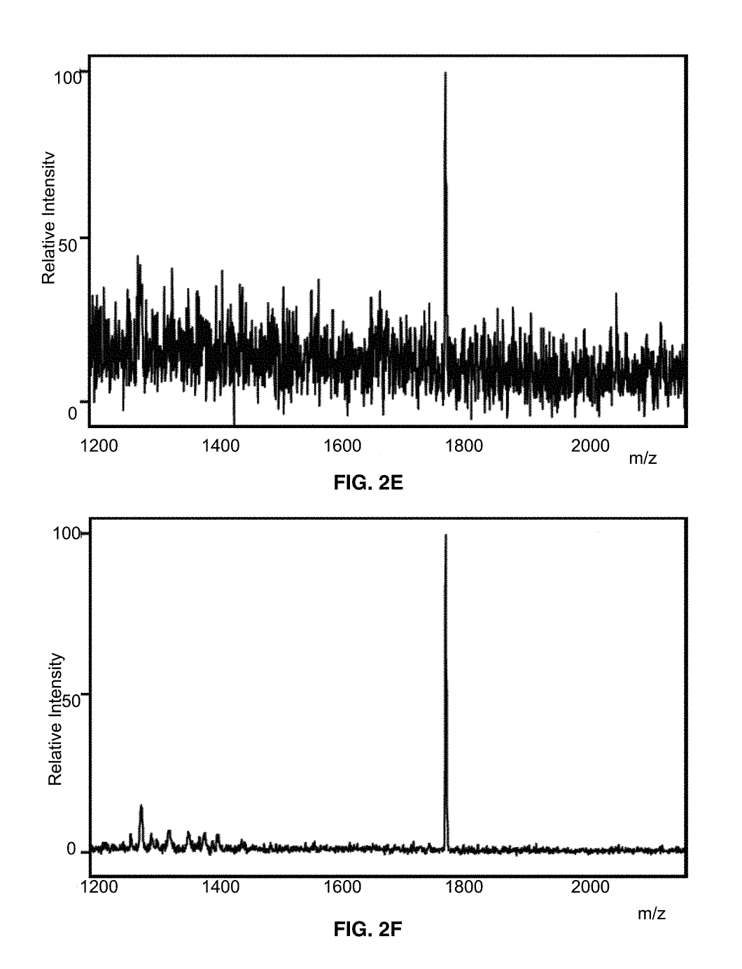

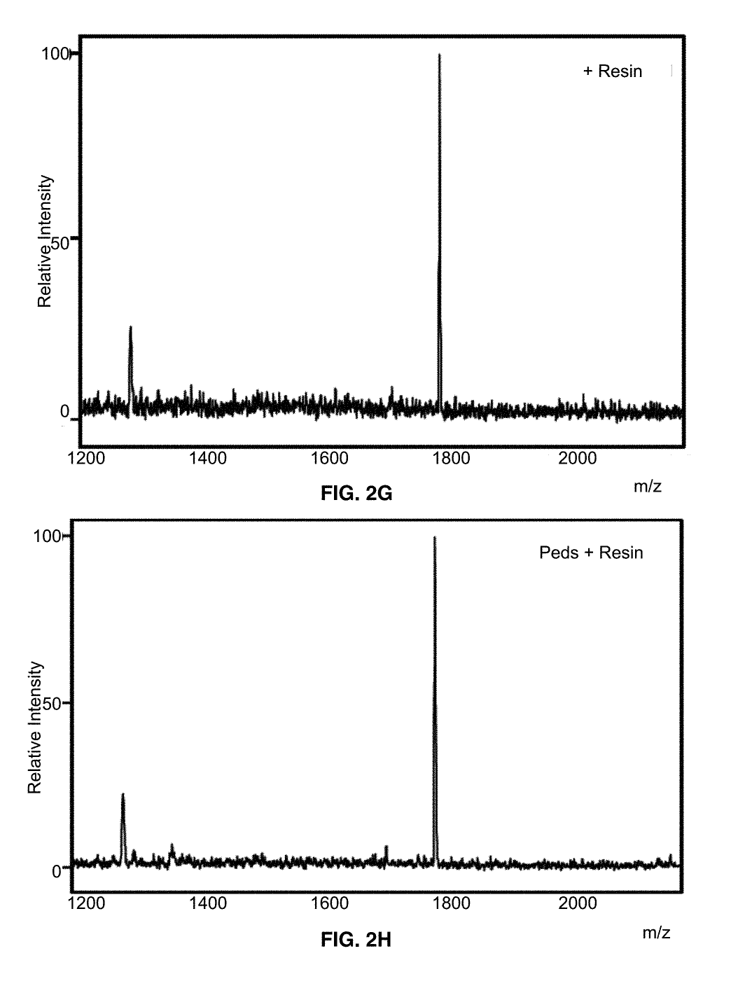

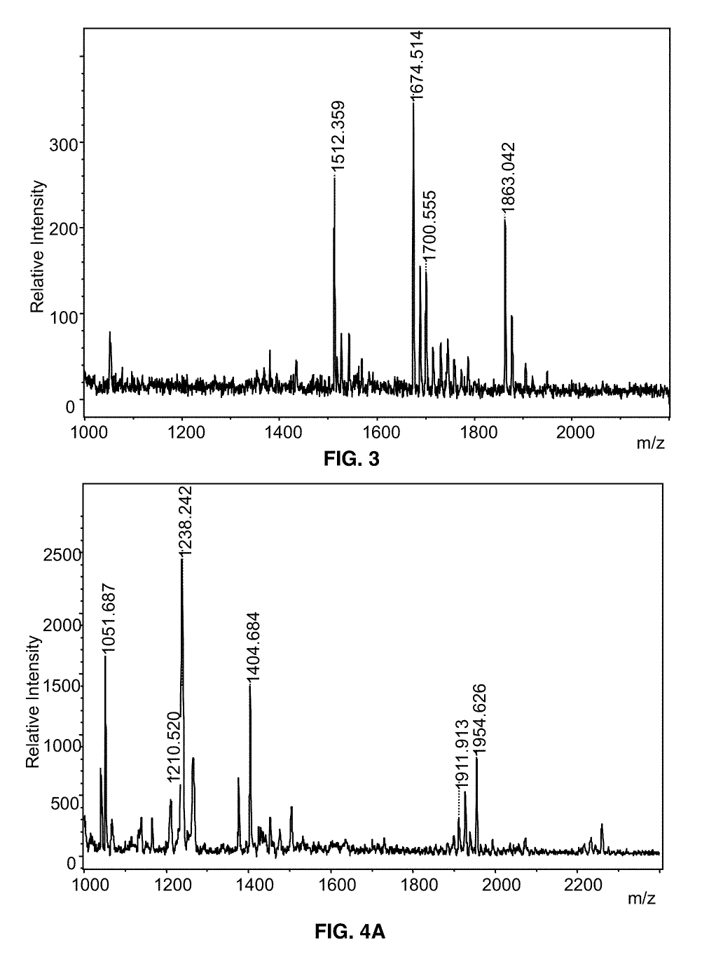

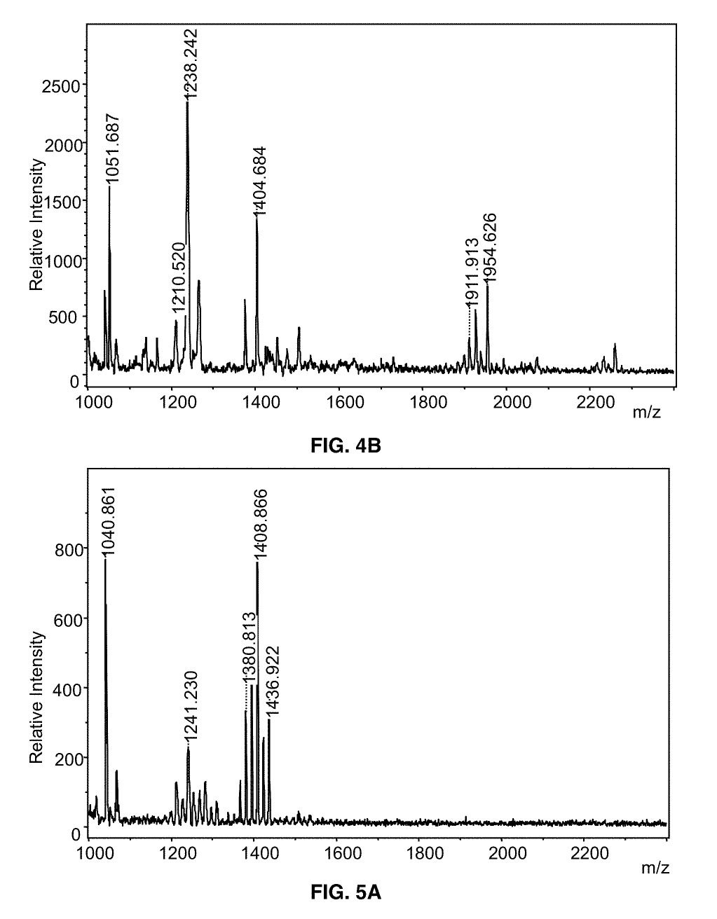

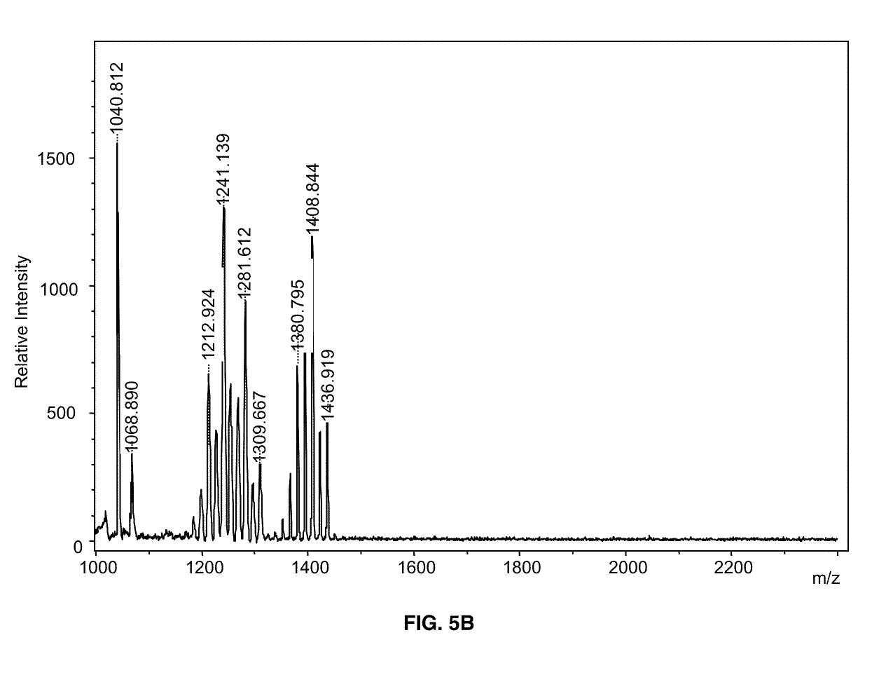

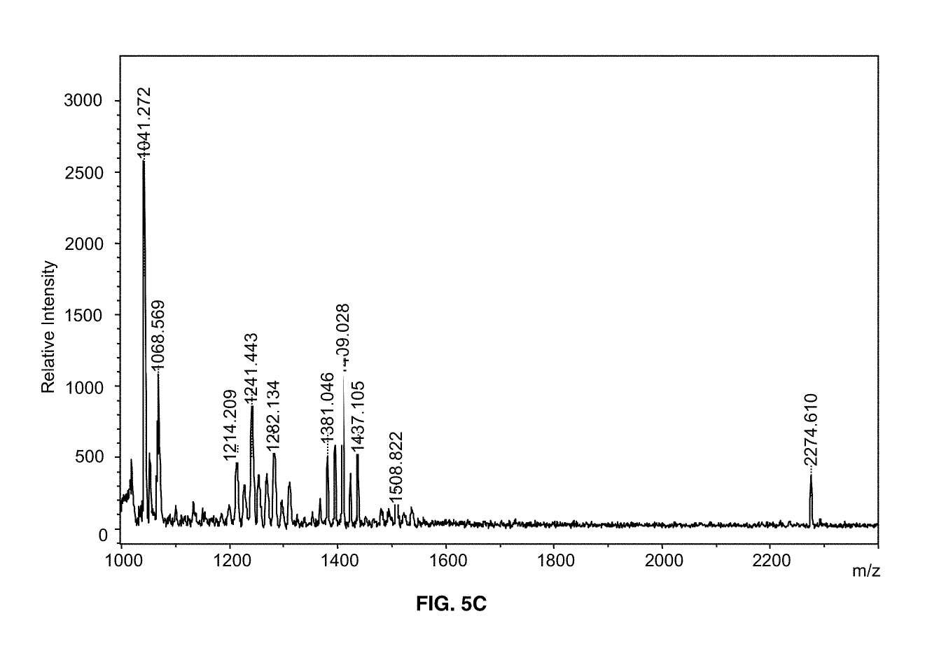

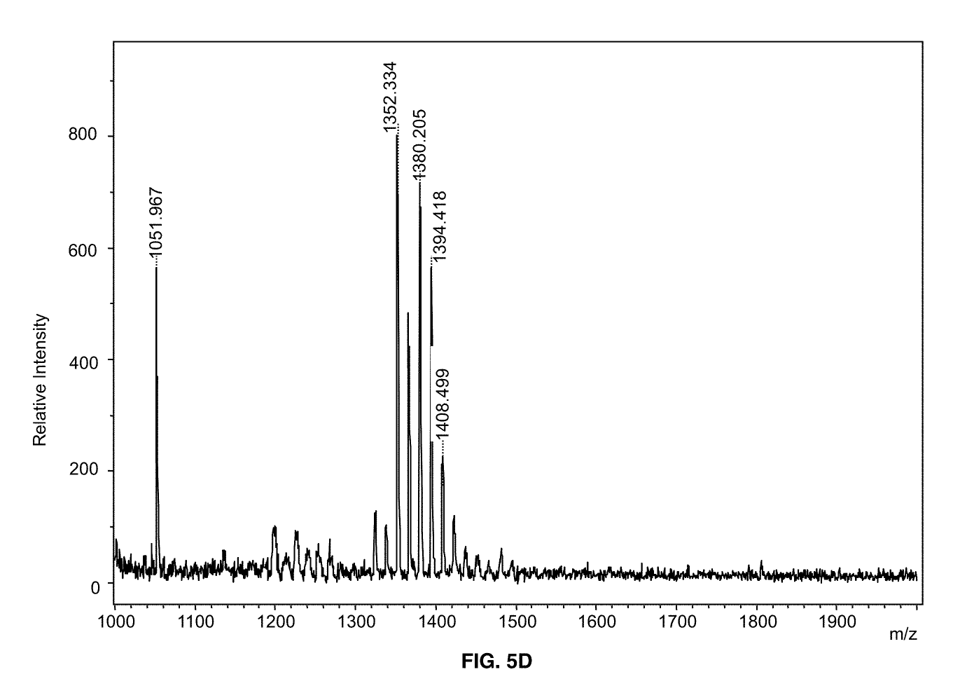

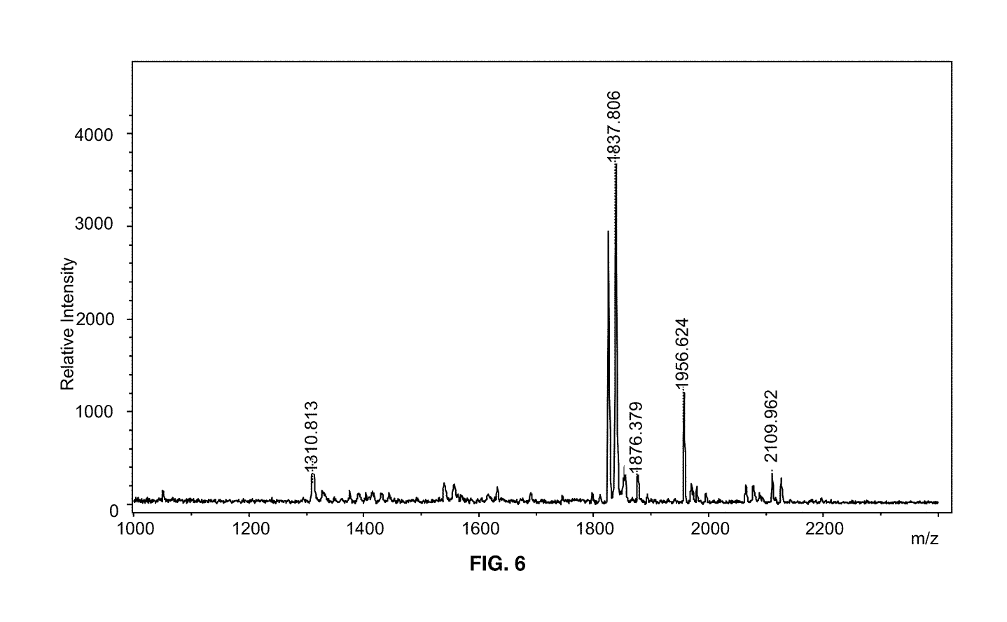

FIG. 1 shows the strategy of mass spectrometric analysis of Escherichia coli lipid A from blood bottles. FIGS. 2A-2H shows MALDI-TOF analysis of Escherichia coli lipid A extracted using differential centrifugation. Analysis of mass spectra was used to demonstrate the ability of lipid A and Lipoteichoic Acid to distinguish not only bacteria, but also antiobiotic resitance (for example, Methicillin-resistant Staphylococcus aureus (MRSA-M2) and Network on Antimicrobial Resistance in Staphylococcus aureus (MRSA-NRS123) FIGS. 5A-5D) and environmental variants (P.aeruginosa; FIG. 3) at high sensitivity, accuracy and specificity. The method of present invention accurately differentiated between different strains of species as shown in FIGS. 5A & 5D the mass spectra of Methicillin-resistant Staphylococcus aureus (MRSA-M2) and Staphylococcus aureus (MRSA-NRS123) shows different mass peak in hundredths and thousandths. The method of the present invention may be used for accurate distinction of species with as few as 10¹ bacterial cells to 10⁸ bacterial cells in the sample (FIGS. 4A-4B; FIGS. 5A-5C). The present method also differentiates antibiotic resistant species Acinetobacter baumannii and Klebsiella pneumoniae which have only subtle structural change in their lipid A (FIGS. 4A-4B & FIG. 6).

### EXAMPLE 2

### Isolation of Microorganisms from Urine and Micro-Extraction of Lipid A and Lipoteichoic Acid for Mass Spectrometry Analysis

Urine specimens are obtained from the University of Pittsburgh Medical Center and processed immediately or stored at −20° C. upon receipt.

### Preparation of 1M Ammonium Hydroxide Solution

Add 3.89 mL of 0.9 g/mL ammonium hydroxide to 96.1 mL of endotoxin free H₂O for 100 mL of 1M Ammonium hydroxide.

### Preparation of Urine Specimens (Day 1)

Transfer 5-10 mL of the specimen to a 15 mL conical tube and spin the tube at 4000 rpm for 10 minutes. This pellets the cells. Discard the supernatant. Pellets are frozen at −20° C. or are extracted immediately.

### Ammonium Isobutyrate Extraction of Glycolipids (Day 1)

Thaw the pellet and resuspend the wet pellet with 400 μL of a 5:3, v/v, 70% isobutyric acid:1M ammonium hydroxide solution. Transfer to a 1.7 mL screw-cap tube (250 μL of isobutyric acid+150 μL Ammonium hydroxide). Incubate at 100° C. for 30-45 minutes with vortexing every ˜15 min (performed in a fume hood). Cool the tube on ice and centrifuge for at 2,000×g for 15 min. Remove the supernatant to a new tube with 400 μL endotoxin free water (1:1 v/v), put on a surrogate cap with a hole poked into the top to allow sublimation, then freeze and lyophilize overnight. Lyophilization generates a fluffy, white pellet (1).

### Processing of Extracts and MS Analysis (Day 2)

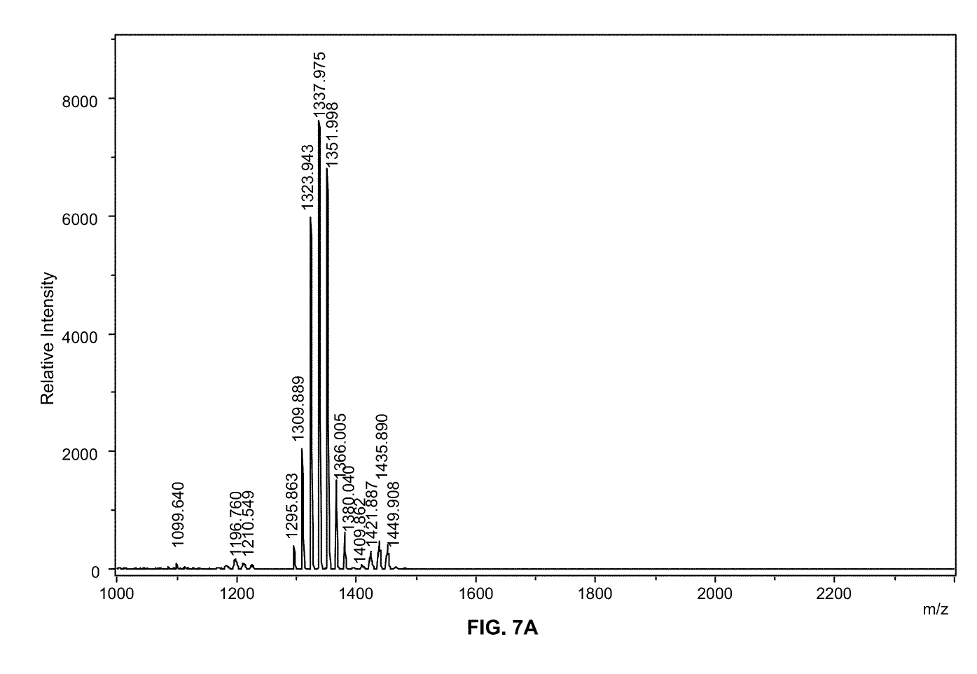

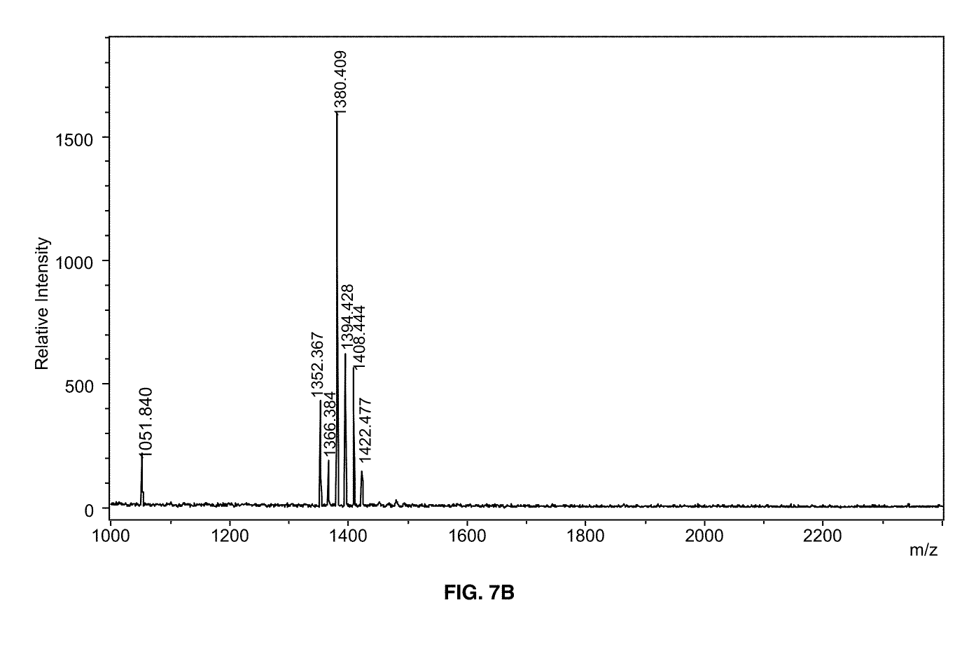

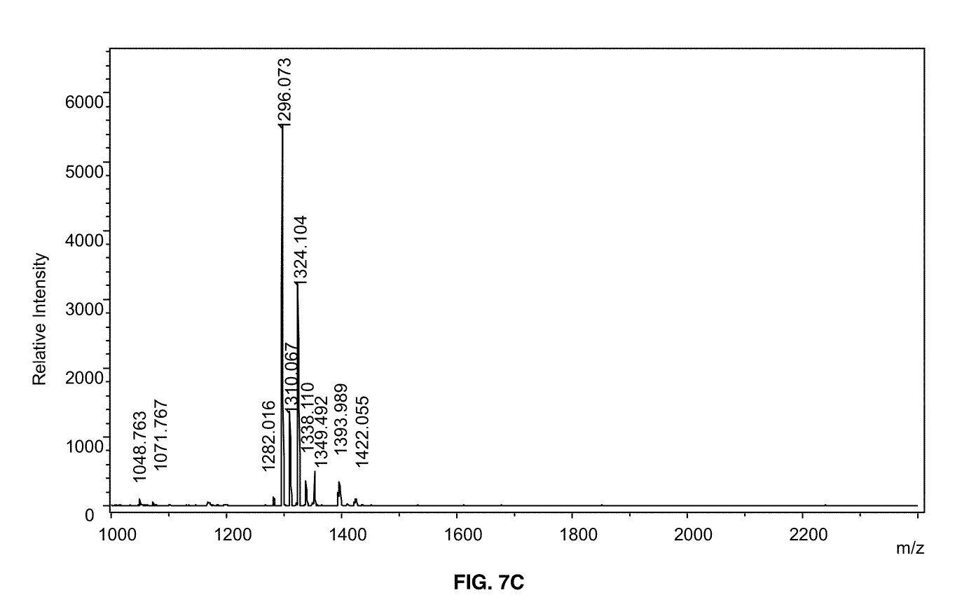

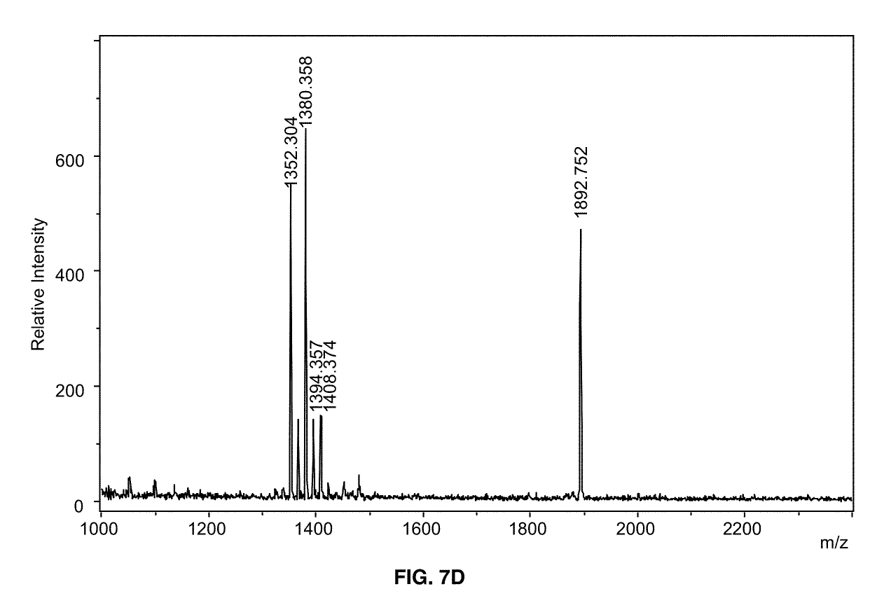

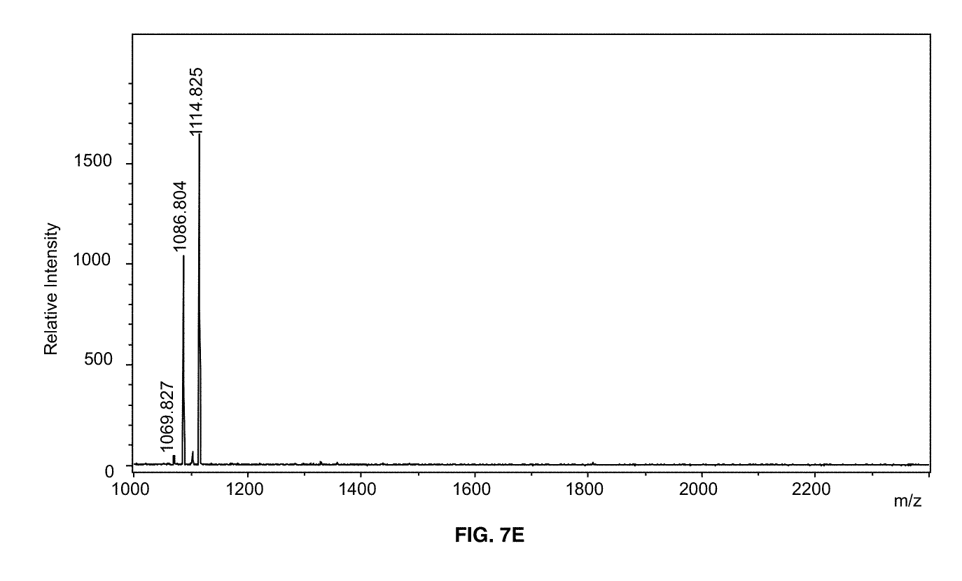

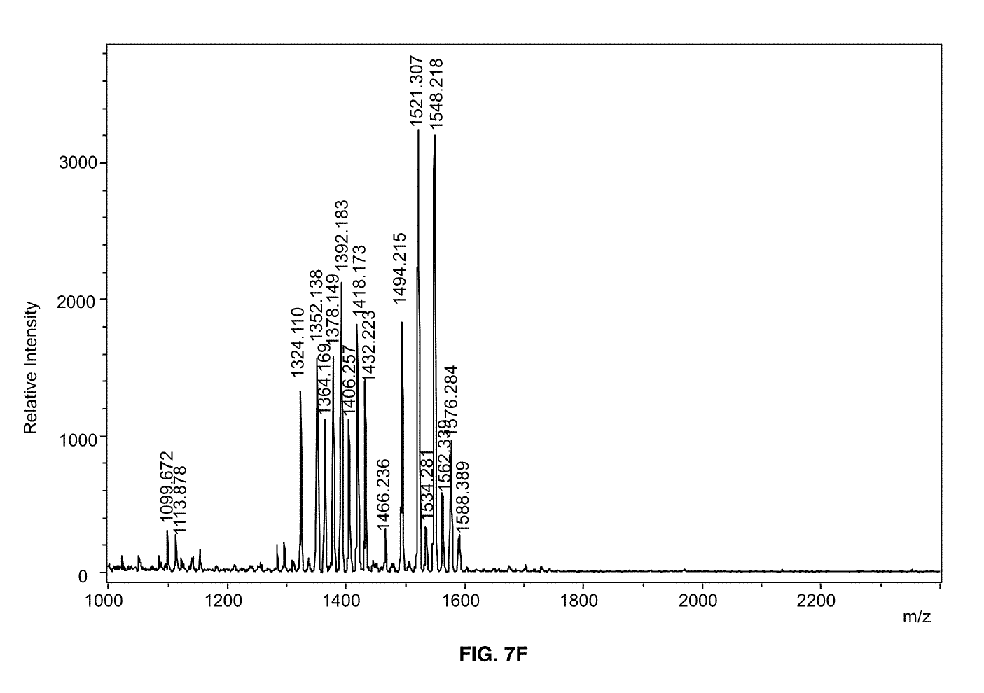

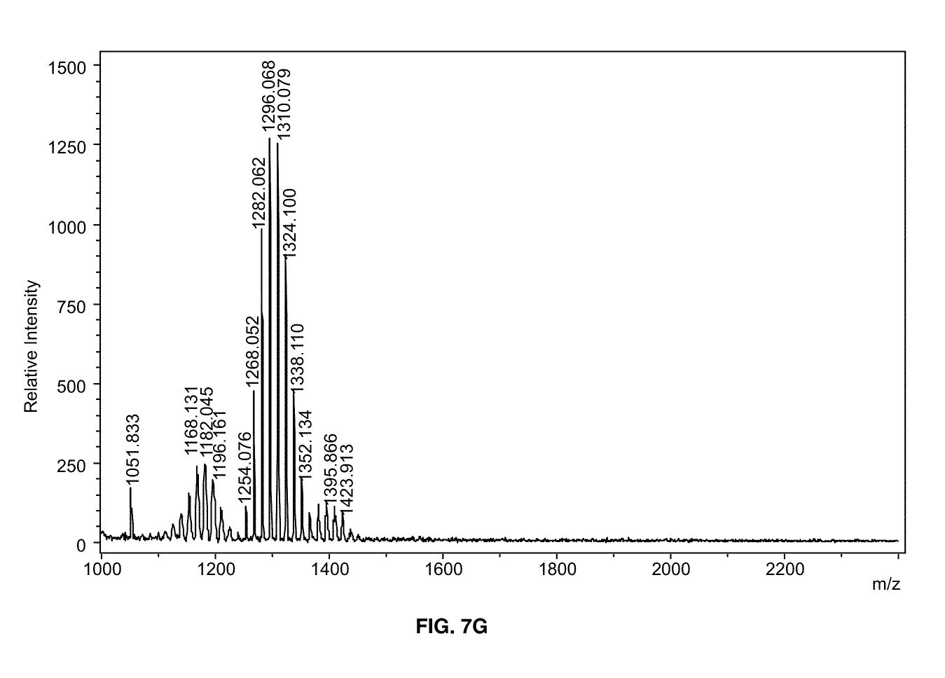

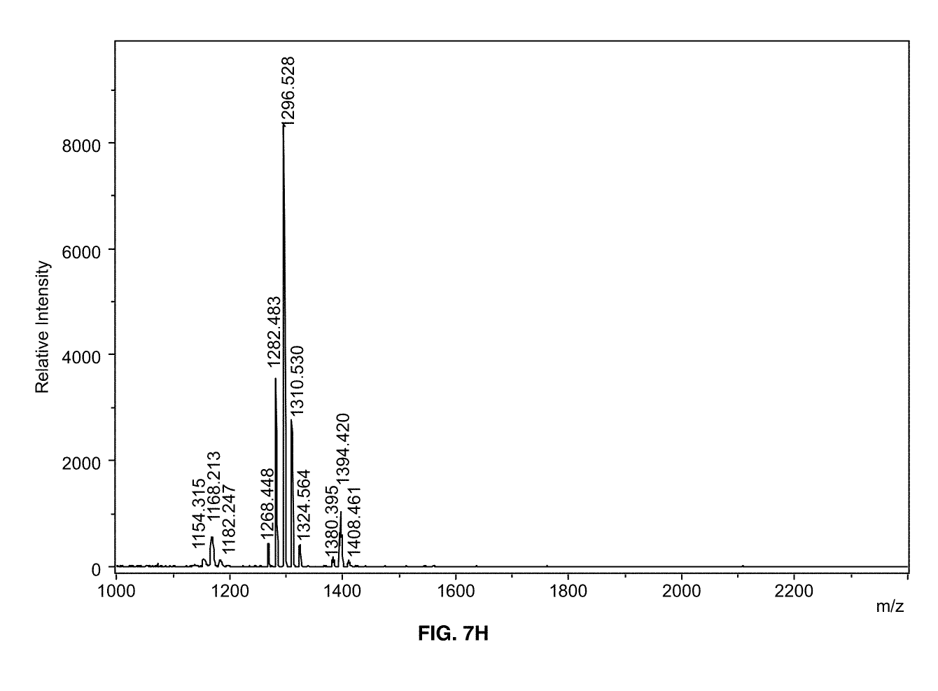

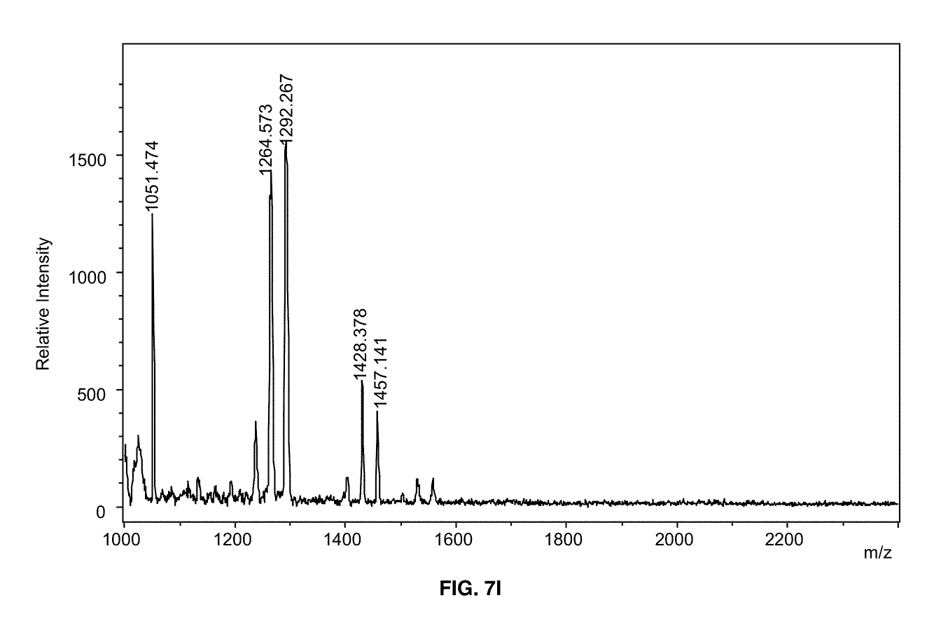

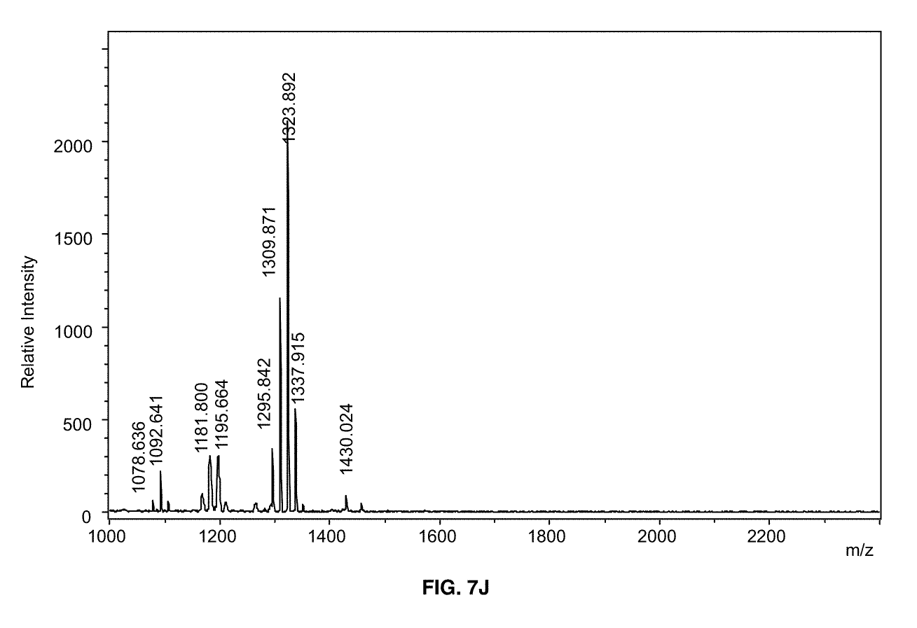

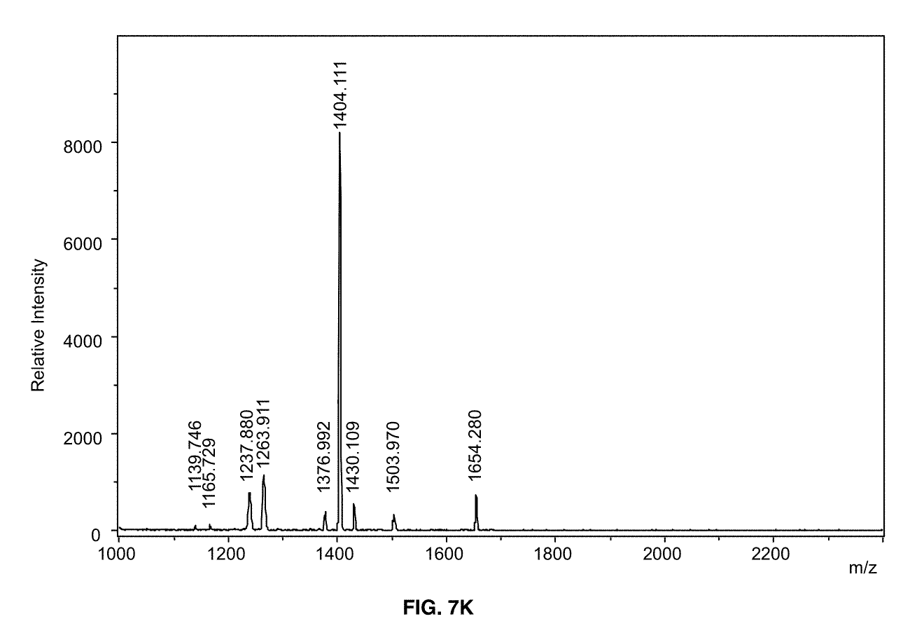

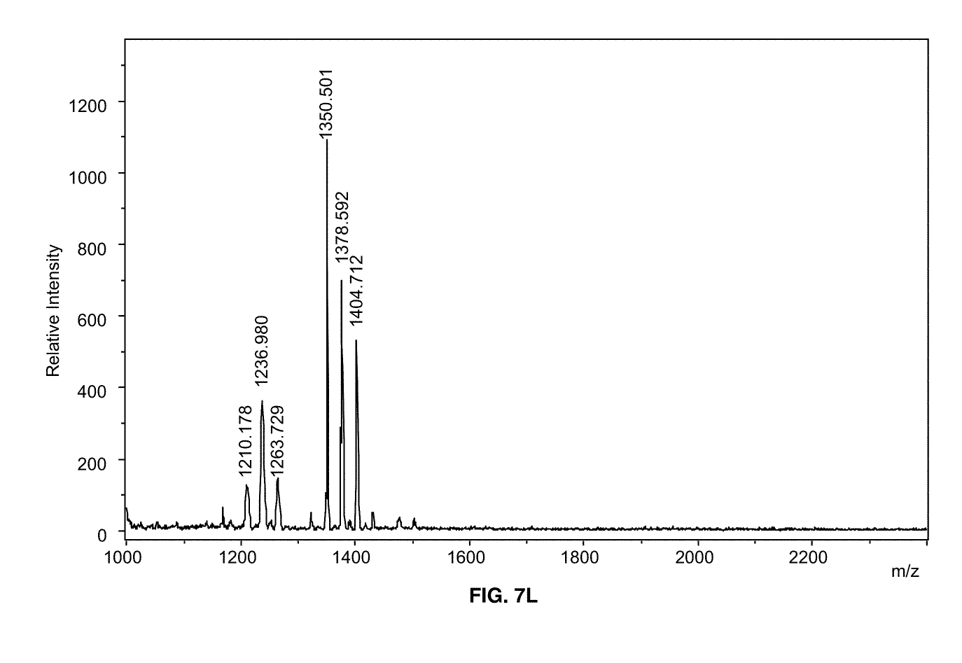

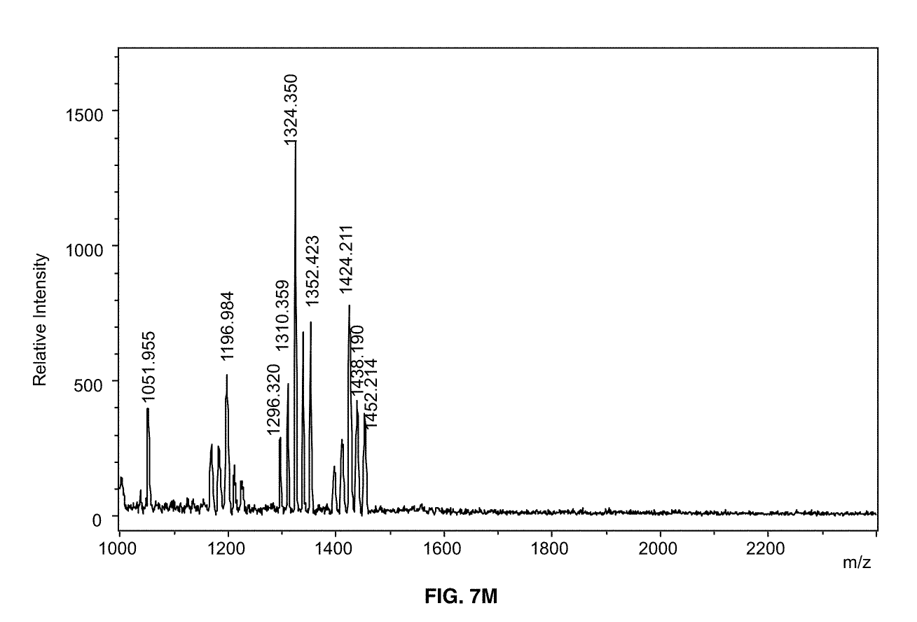

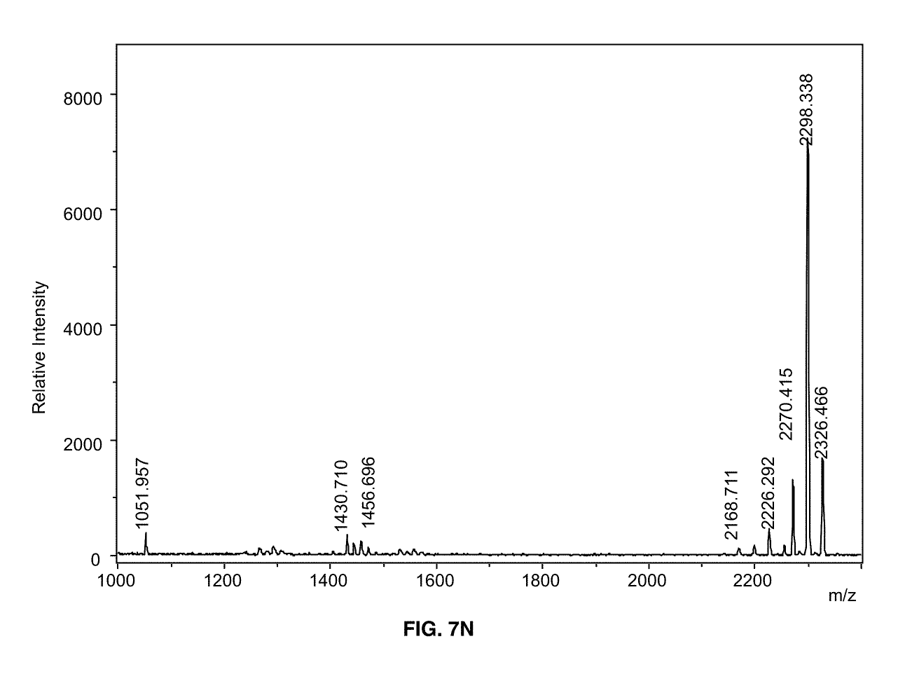

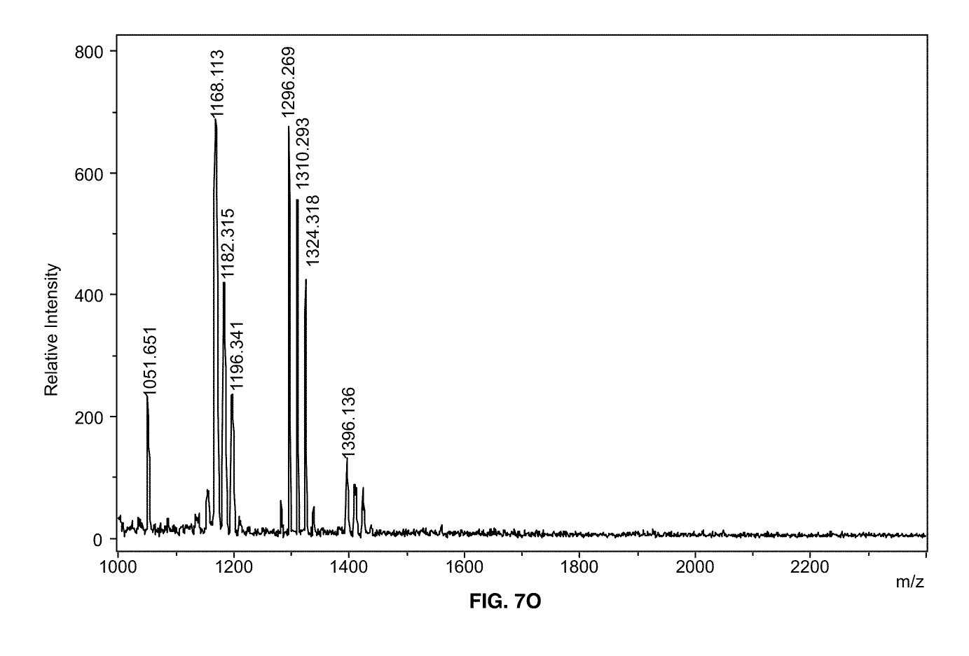

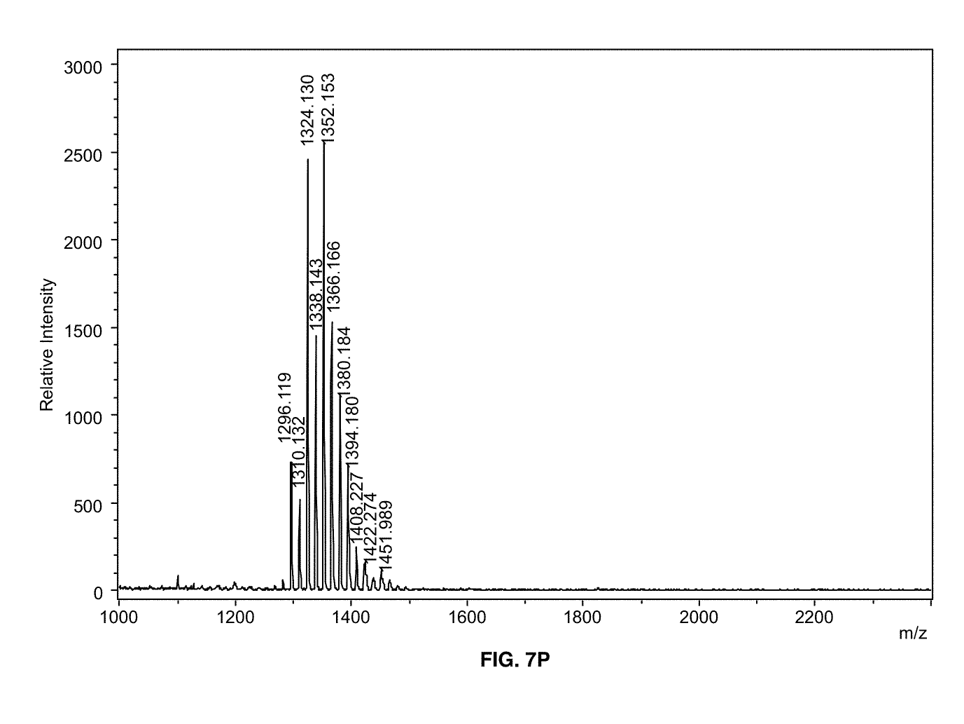

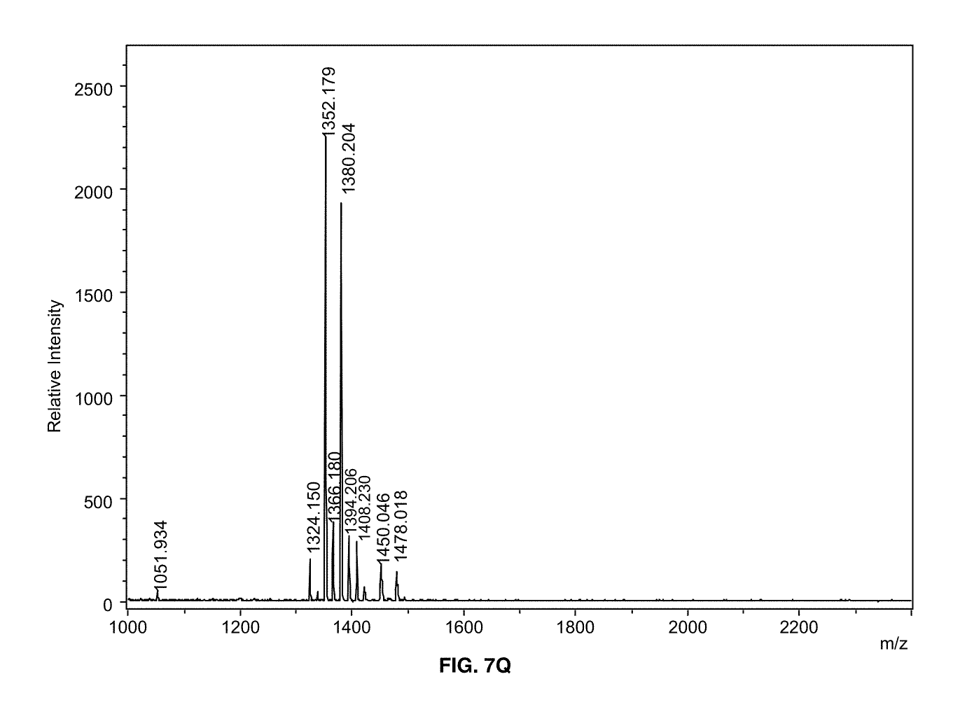

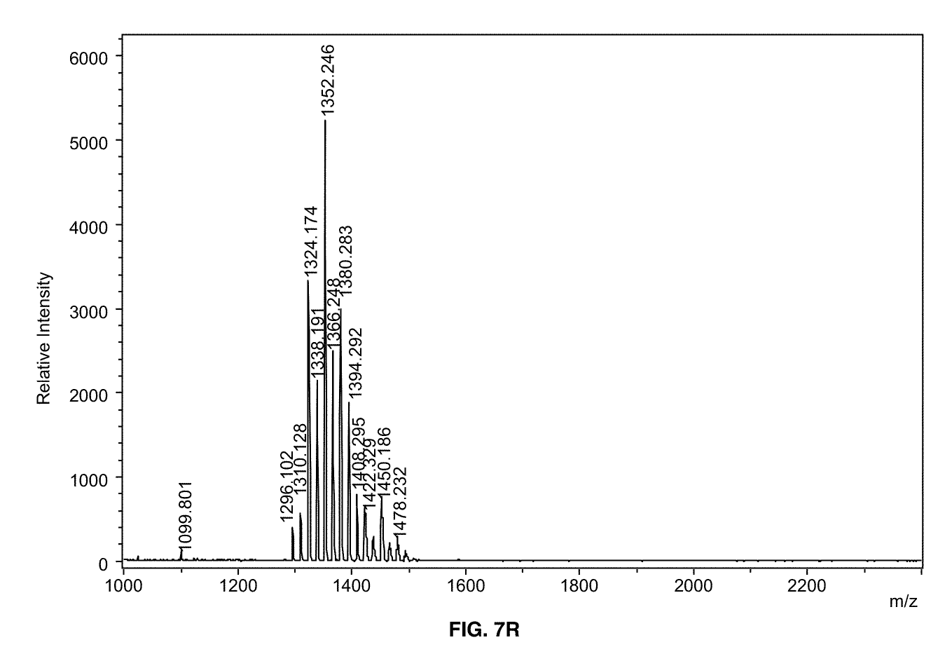

Wash the sample with 1 mL of methanol, sonicate to resuspend and to disrupt the micelles, 5 minutes or more. Centrifuge at 10,000×g for 5 minutes, carefully aspirate the methanol. Wash again with methanol. Solubilize and extract Lipid A in 25-190 μL of 3:1.5:0.25, v/v/v, chloroform:methanol:water, i.e., 120/60/10 μL. Vortex well. Optionally add ˜5 grains of Dowex 50WX8 (H+) and vortex well to desalt (e.g. Ca2+, Mg2+, and Na+). This enhances negative mode MALDI-MS sensitivity. Vortex and centrifuge at 8,000×g for 5 minutes. Spot 1 μL on a MALDI target plate, followed by 1 μL fresh matrix, e.g., 10 [mg/mL] norharmane in 2:1, v/v, chloroform:methanol. Mass spectra are recorded in negative ion mode at 60-70% laser intensity and 900 laser shots per spectrum using a Bruker Microflex LRF MALDI-TOF (2) (FIGS. 7A-7R).

FIGS. 7A-7R shows representative examples of mass spectra of closely related Gram-positive bacteria, Gram-negative bacteria and fungus, differentiating them on the basis of different molecular mass profile of lipid A, Lipoteichoic Acid or glycolipid in urine sample. The method of present invention was used to accurately differentiate between closely related species of, for example, Pseudomonas and Staphylococcus in urine sample. The mass analysis accurately differentiated the lipid A mass profile of gram negative Pseudomonas oryzihabitans and Pseudomonas stutzeri, both having different mass peaks (FIGS. 7K-7L). Similarly, Gram-positive Staphylococcus species were differentiated by their different Lipoteichoic Acid mass profile (FIGS. 7P-7R).

### EXAMPLE 3

### Isolation of Microorganisms from Feces and Micro-Extraction of Lipid A and Lipoteichoic Acid for Mass Spectrometry Analysis

Murine fecal specimens are obtained from the lab of Dr. Hanping Feng at the University of Maryland, Baltimore and are processed immediately or are stored at −20° C. upon receipt.

### Preparation of 1M Ammonium Hydroxide Solution

Add 3.89 mL of 0.9 g/mL ammonium hydroxide to 96.1 mL of endotoxin free H₂O for 100 mL of 1M ammonium hydroxide.

### Preparation of Fecal Specimens (Day 1)

Transfer the specimen to a 2 mL sterile microcentrifuge tube and suspend it in 1×PBS. Don't fill the tube past 1 mL of volume. Use a tissue grinder pestle to disrupt the feces and to generate a slurry and spin the tube at 1100 rpm for 10 minutes. This pellets fecal debris and human cells. Transfer the supernatant to a 1.7 mL screw-cap tube and spin the tube at 4000 rpm for 10 minutes. This pellets the bacterial cells. Discard the supernatant. Pellets are frozen at −20° C. or are extracted immediately.

### Ammonium Isobutyrate Extraction of Glycolipids (Day 1)

Thaw the pellet and resuspend the wet pellet with 400 μL of a 5:3, v/v, 70% isobutyric acid:1M ammonium hydroxide solution. Transfer to a 1.7 mL screw-cap tube (250 μL of isobutyric acid+150 μL ammonium hydroxide). Incubate at 100° C. for 30-45 minutes with vortexing every ˜15 min (performed in a fume hood). Cool the tube on ice and centrifuge at 2,000×g for 15 min. Remove the supernatant to a new tube with 400 μL endotoxin free water (1:1 v/v), put on a surrogate cap with a hole poked into the top to allow sublimation, freeze and lyophilize overnight. Lyophilization generates a fluffy, white pellet (1).

### Processing of Extracts and MS Analysis (Day 2)

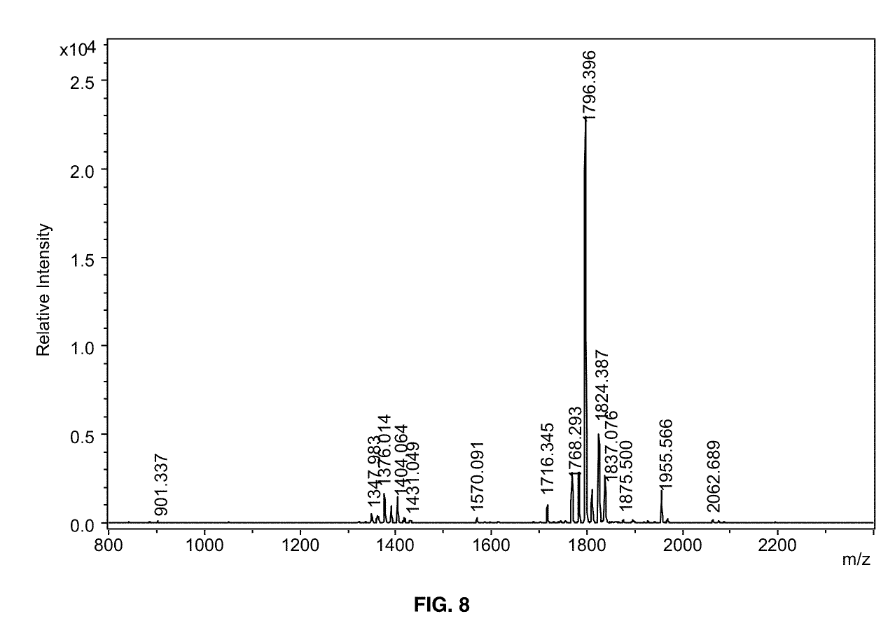

Wash the sample with 1 mL of methanol, sonicate to resuspend and to disrupt the micelles for 5 minutes or more. Centrifuge at 10,000×g for 5 minutes and carefully aspirate the methanol. Wash again with methanol. Solubilize and extract Lipid A in 25-190 μL of 3:1.5:0.25, v/v/v, chloroform:methanol:water, i.e. 120/60/10 μL. Vortex well. Optionally add ˜5 grains of Dowex 50WX8 (H+) and vortex well to desalt (e.g. Ca2+, Mg2+, and Na+). This enhances negative mode MALDI-MS sensitivity. Vortex and centrifuge at 8,000×g for 5 minutes. Spot 1 μL onto the MALDI target plate and follow with 1 μL fresh matrix, e.g. 10 [mg/mL] norharmane in 2:1, v/v, chloroform:methanol). Mass spectra are recorded in negative ion mode at 60-70% laser intensity and 900 laser shots per spectrum using a Bruker Microflex LRF MALDI-TOF (2). FIG. 8 shows the mass spectrometric analysis of E. coli in one fecal pellet incubated overnight in liquid medium.

### EXAMPLE 4

### Isolation of Microorganisms from Wound Effluent (WE), Bronchoalveolar Lavage Fluid (BAL), Serum and Micro-extraction of Lipid A and Lipoteichoic Acid for Mass Spectrometry Analysis

### Preparation of 1M Ammonium Hydroxide Solution

Add 3.89 mL of 0.9 g/mL ammonium hydroxide to 96.1 mL of endotoxin free H₂O for 100 mL of 1M ammonium hydroxide.

### Preparation of Wound Effluent, BAL, and Serum Specimens (Day 1)

Transfer 5-10 mL of the specimen to a 15 mL conical tube and spin the tube at 4000 rpm for 10 minutes. This pellets the cells. Discard the supernatant. Pellets are frozen at −20° C. or are extracted immediately.

### Ammonium Isobutyrate Extraction of Glycolipids (Day 1)

Thaw the pellet and resuspend the wet pellet with 400 μL of a 5:3, v/v, 70% isobutyric acid:1M ammonium hydroxide solution. Transfer to a 1.7 mL screw-cap tube (250 μL of isobutyric acid+150 μL Ammonium hydroxide). Incubate at 100° C. for 30-45 minutes with vortexing every ˜15 min (performed in a fume hood). Cool the tube on ice and centrifuge for at 2,000×g for 15 min. Remove the supernatant to a new tube with 400 μL endotoxin free water (1:1 v/v), put on a surrogate cap with a hole poked into the top to allow sublimation, freeze and lyophilize overnight. Lyophilization generates a fluffy, white pellet (1).

### Processing of Extracts and MS Analysis (Day 2)

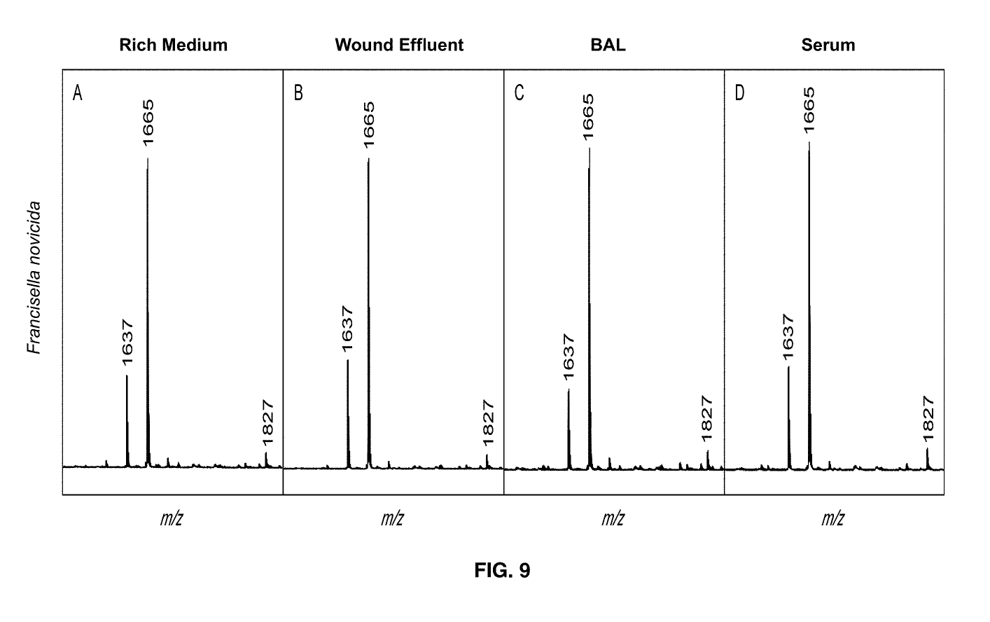

Wash the sample with 1 mL of methanol, sonicate to resuspend and to disrupt micelles for 5 minutes or more. Centrifuge at 10,000×g for 5 minutes and carefully aspirate the methanol. Wash again with methanol. Solubilize and extract Lipid A in 25-190 μL of 3:1.5:0.25, v/v/v, chloroform:methanol:water, i.e. 120/60/10 μL. Vortex well. Optionally add ˜5 grains of Dowex 50WX8 (H+) and vortex well to desalt (e.g. Ca2+, Mg2+, and Na+). This enhances negative mode MALDI-MS sensitivity Vortex and centrifuge at 8,000×g for 5 minutes. Spot 1 μL onto the MALDI target plate and follow with 1 μL fresh matrix, e.g. 10 [mg/mL] norharmane in 2:1, v/v, chloroform:methanol) Mass spectra are recorded in negative ion mode at 60-70% laser intensity and 900 laser shots per spectrum using a Bruker Microflex LRF MALDI-TOF (2). FIG. 9 shows the mass spectrometric analysis of Francisella species done in rich medium, wound effluent, bronchoalveolar lavage fluid and serum.

### EXAMPLE 5

### Pathogenic Strains

Referring to Table 1 below, 520 isolates of pathogens were analyzed by the molecular mass profile of the lipids via mass spectrometry. The reference organisms presented in Table 1 include 32 of the most common bacterial species, 1 fungal species isolated from biological samples and 6 bacterial strains isolated from blood samples.

TABLE 1
LIST OF SPECIES FOR MS LIPID ANALYSIS

| Species | Strain | Species | Strain |
|---|---|---|---|
| Enterococcus faecalis | FN1 | Klebsiella pneumoniae | F1 |
| Enterococcus faecalis | FN2 | Klebsiella pneumoniae | S804263 |
| Enterococcus faecalis | FN14 | Klebsiella pneumoniae | T1173546 |
| Enterococcus faecalis | FN39 | Klebsiella pneumoniae | F28006 |
| Enterococcus faecalis | FN45 | Klebsiella pneumoniae | S7233 |
| Enterococcus faecalis | FN46 | Klebsiella pneumoniae | F87280 |
| Enterococcus faecalis | FN59 | Klebsiella pneumoniae | F3292636 |
| Enterococcus faecalis | FN69 | Klebsiella pneumoniae | X30374 |
| Enterococcus faecalis | FN71 | Klebsiella pneumoniae | M48525 |
| Enterococcus faecalis | FN77 | Klebsiella pneumoniae | S81517 |
| Enterococcus faecium | M32517 | Klebsiella pneumoniae | S83925 |
| Enterococcus faecium | X7357 | Klebsiella pneumoniae | H796922 |
| Enterococcus faecium | T35657 | Klebsiella pneumoniae | F900627 |
| Enterococcus faecium | T23567 | Klebsiella pneumoniae | S775008 |
| Enterococcus faecium | W28461 | Klebsiella pneumoniae | T1313002 |
| Enterococcus faecium | W5454959 | Klebsiella pneumoniae | S1428956 |
| Enterococcus faecium | T5504515 | Klebsiella pneumoniae | W2034211 |
| Enterococcus faecium | S3899836 | Klebsiella pneumoniae | A2 Obscure |
| Enterococcus faecium | H16150 | Klebsiella pneumoniae | B3 Bright |
| Enterococcus faecium | M37969 | Klebsiella pneumoniae | B3 Obscure |
| Staphylococcus aureus | NRS22 | Klebsiella pneumoniae | B5 |
| Staphylococcus aureus | NRS387 | Klebsiella pneumoniae | B8 |
| Staphylococcus aureus | NRS384 | Klebsiella pneumoniae | C4 |
| Staphylococcus aureus | NRS382 | Klebsiella pneumoniae | D4 |
| Staphylococcus aureus | NRS1 | Klebsiella pneumoniae | D7 |
| Staphylococcus aureus | M2 | Klebsiella pneumoniae | E5 |
| Staphylococcus aureus | NRS123 | Klebsiella pneumoniae | F28006 |
| Staphylococcus aureus | NRS385 | Klebsiella pneumoniae | F8 |
| Staphylococcus aureus | NRS100 | Klebsiella pneumoniae | G7 |
| Staphylococcus aureus | NRS484 | Klebsiella pneumoniae | H4 Bright |
| Staphylococcus aureus | NRS72 | Klebsiella pneumoniae | I1 |
| Staphylococcus aureus | RN6390 | Klebsiella pneumoniae | A5 |
| Staphylococcus aureus | Seattle 1945 | Klebsiella pneumoniae | B6 |
| Staphylococcus aureus | RN4220 | Klebsiella pneumoniae | B9 |
| Staphylococcus aureus | 8325-4 | Klebsiella pneumoniae | C1 |
| Staphylococcus aureus | M1 | Klebsiella pneumoniae | C5 |
| Klebsiella pneumoniae | F645516 | Klebsiella pneumoniae | C6 |
| Klebsiella pneumoniae | F892412 | Klebsiella pneumoniae | C8 |
| Klebsiella pneumoniae | T1019279 | Klebsiella pneumoniae | D1 |
| Klebsiella pneumoniae | S777647 | Klebsiella pneumoniae | F1 |
| Klebsiella pneumoniae | E2 | Acinetobacter baumannii | ATCC C2B |
| Klebsiella pneumoniae | E6 | Acinetobacter baumannii | ATCC 17978 WT |
| Pseudomonas putida | 6732-1 oxidative (−) | Acinetobacter baumannii | AB0057 |
| Klebsiella pneumoniae | F3292636 | Acinetobacter baumannii | M537095; ColS |
| Klebsiella pneumoniae | F645516 | Acinetobacter baumannii | S1444428; ColS |
| Klebsiella pneumoniae | F900627 | Acinetobacter baumannii | 2B3; H1905339 |
| Klebsiella pneumoniae | H5 | Acinetobacter baumannii | 2C8; T1316530 |
| Klebsiella pneumoniae | I2 | Acinetobacter baumannii | 1I5; X1329321 |
| Klebsiella pneumoniae | I4 | Acinetobacter baumannii | 1I6; S1259244 |
| Klebsiella pneumoniae | I6 | Acinetobacter baumannii | 2A7; W1910338 |
| Escherichia coli | ATCC 43888 | Salmonella minnesota | R595; ATCC |
| | | | 49284 |
| Salmonella typhimurium | CS339 | Pseudomonas aeruginosa | BE-174 |
| Burkholderia cenocepacia | ATCC 17759 | Pseudomonas aeruginosa | BE-175 |
| Genomovar I | | | |
| Francisella novicida | U112 | Pseudomonas aeruginosa | BE-176 |
| Escherichia coli | BW25113 | Pseudomonas aeruginosa | BE-177 |
| Pseudomonas aeruginosa | PAK | Pseudomonas aeruginosa | BE-178 |
| Burkholderia cenocepacia | CEP0790 | Pseudomonas aeruginosa | BE-398 |
| Burkholderia multivorans | ATCC 17616 | Pseudomonas aeruginosa | BE-399 |
| Genomovar II | | | |
| Pseudomonas aeruginosa | H35N::PA1393 | Pseudomonas aeruginosa | BE-400 |
| Pseudomonas putida | ATCC 700007 | Pseudomonas aeruginosa | BE-401 |
| Stenotrophomonas | CF 2 | Pseudomonas aeruginosa | BE-402 |
| maltophilia | | | |
| Yersinia pestis | KIM6−pCDI−pgm− | Enterobacter cloacae | FN2462 |
| Yersinia pestis | KIM6+ (Bliska) | Enterobacter cloacae | FN2468 |
| Francisella tularensis | LVS | Enterobacter cloacae | FN2475 |
| holarctica | | | |
| Pseudomonas fluorescens | ATCC BAA-477 | Enterobacter cloacae | FN2486 |
| Burkholderia cenocepacia | ATCC 17616 | Enterobacter cloacae | FN2531 |
| Genomovar II | | | |
| Pseudomonas aeruginosa | H35N::PA1393 | Enterobacter cloacae | FN2532 |
| Pseudomonas putida | ATCC 700007 | Enterobacter cloacae | FN2540 |
| Stenotrophomonas | CF 2 | Enterobacter cloacae | FN2541 |
| maltophilia | | | |
| Yersinia pseudotuberculosis | 01:b | Enterobacter cloacae | FN2542 |
| Yersinia enterocolitica | CS080 | Enterobacter cloacae | FN2543 |
| Acinetobacter baumannii | ATCC 19606 | Enterobacter cloacae | YDC469-1 |
| | WT colistin- | | |
| | sensitive | | |
| Acinetobacter baumannii | ATCC 19606 | Enterobacter cloacae | YDC476 |
| | WT colistin- | | |
| | resistant | | |
| Enterobacter cloacae | YSC506 | Acinetobacter baumannii | M6142110 |
| Enterobacter cloacae | YDC567 | Acinetobacter baumannii | M6154841 |
| Enterobacter cloacae | YDC572 | Acinetobacter baumannii | T6236674 |
| Enterobacter cloacae | YDC590 | Acinetobacter baumannii | T6276391 # 1 |
| Acinetobacter baumannii | T25987; ColS | Acinetobacter baumannii | T6276391 # 2 |
| Enterobacter cloacae | YDC603 | Acinetobacter baumannii | T6262055 |
| Enterobacter cloacae | YDC612 | Acinetobacter baumannii | S4372736 # 1 |
| Enterobacter cloacae | YDC665 | Acinetobacter baumannii | S4372736 # 2 |
| Enterobacter cloacae | YDC673 | Acinetobacter baumannii | T6292796 |
| Acinetobacter baumannii | 1H7; S906365 | Acinetobacter baumannii | W6231191 |
| Acinetobacter baumannii | 1G5; F1918631 | Acinetobacter baumannii | H6195648 |
| Acinetobacter baumannii | 1I7; W18065482 | Acinetobacter baumannii | W6248513 |
| Acinetobacter baumannii | 2B9; T2796953 | Acinetobacter baumannii | F6001181 |
| Acinetobacter baumannii | 2G3 | Acinetobacter baumannii | F6005058 |
| Acinetobacter baumannii | 2A8; H1883446 | Acinetobacter baumannii | S4409585 |
| Acinetobacter baumannii | C8 | Acinetobacter baumannii | T6345606 |
| Acinetobacter baumannii | MU181 | Acinetobacter baumannii | W6265964 |
| Acinetobacter baumannii | MU215 | Acinetobacter baumannii | T6337518 |
| Pseudomonas aeruginosa | PAO1 | Acinetobacter baumannii | X3967941 |
| Acinetobacter baumannii | S4259384 | Acinetobacter baumannii | T6374080 |
| Acinetobacter baumannii | S4249014 | Acinetobacter baumannii | M6307212 |
| Acinetobacter baumannii | M6139359 | Serratia marcescens | SM 3 |
| Acinetobacter baumannii | S4217436 | Serratia marcescens | SM 4 |
| Acinetobacter baumannii | H6076944 | Serratia marcescens | SM 5 |
| Acinetobacter baumannii | M6138054 | Serratia marcescens | SM 8 |
| Acinetobacter baumannii | H6137766 | Serratia marcescens | SM 9 |
| Acinetobacter baumannii | S4393349 | Serratia marcescens | SM 11 |
| Acinetobacter baumannii | M6226989 | Serratia marcescens | SM 12 |
| Acinetobacter baumannii | F5727811 | Serratia marcescens | SM 13 |
| Acinetobacter baumannii | M6004145 | Serratia marcescens | M6315510 |
| Acinetobacter baumannii | X3812952 | Serratia marcescens | X3925583 # 1 |
| Acinetobacter baumannii | F5832616 | Serratia marcescens | X3925583 # 2 |
| Acinetobacter baumannii | F5835440 | Acinetobacter baumannii | T6542349 |
| Acinetobacter baumannii | S4292042 | Acinetobacter baumannii | H6432894 |
| Acinetobacter baumannii | X3845805 | Acinetobacter baumannii | M6324995 |
| Acinetobacter baumannii | T6172219 | Acinetobacter baumannii | H6343630 |
| Acinetobacter baumannii | H6078005 | Acinetobacter baumannii | S4510581 |
| Acinetobacter baumannii | F5852249 | Acinetobacter baumannii | X4075444 |
| Acinetobacter baumannii | F5847155 | Acinetobacter baumannii | F6201782 |
| Acinetobacter baumannii | M6113993 | Acinetobacter baumannii | H6468736 # 1 |
| Acinetobacter baumannii | H6094261 | Acinetobacter baumannii | H6468736 # 2 |
| Acinetobacter baumannii | S4326066 | Acinetobacter baumannii | H6509069 |
| Acinetobacter baumannii | H6504483 | Acinetobacter baumannii | H6668538 |
| Acinetobacter baumannii | F6275094 | Acinetobacter baumannii | F6413360 |
| Acinetobacter baumannii | W6371925 | Acinetobacter baumannii | H6689003 # 1 |
| Acinetobacter baumannii | S4489171-1 | Acinetobacter baumannii | H6689003 # 2 |
| Acinetobacter baumannii | M6368166 | Acinetobacter baumannii | H6688979 |
| Acinetobacter baumannii | W6393209 | Acinetobacter baumannii | F6435319 # 1 |
| Acinetobacter baumannii | M6391197 | Acinetobacter baumannii | F6435319 # 2 |
| Acinetobacter baumannii | T6520248 | Acinetobacter baumannii | S4715753 |
| Acinetobacter baumannii | H6417812 | Acinetobacter baumannii | S4715756 |
| Acinetobacter baumannii | S4542196 | Acinetobacter baumannii | S4720954 |
| Acinetobacter baumannii | T6530325 | Acinetobacter baumannii | M6716608 |
| Acinetobacter baumannii | H6449923 | Acinetobacter baumannii | W6741369 |
| Acinetobacter baumannii | H6465612 # 1 | Acinetobacter baumannii | H6708536 |
| Acinetobacter baumannii | H6465612 # 2 | Acinetobacter baumannii | T6837121 |
| Acinetobacter baumannii | H6467111 | Acinetobacter baumannii | T6844157 |
| Acinetobacter baumannii | F6212404 | Acinetobacter baumannii | W6788536 |
| Acinetobacter baumannii | X4108289 | Acinetobacter baumannii | W6788537 |
| Acinetobacter baumannii | F6248736 | Acinetobacter baumannii | W6798196 |
| Acinetobacter baumannii | H6522237 | Acinetobacter baumannii | T6912170 |
| Acinetobacter baumannii | F6259710 | Acinetobacter baumannii | S4820321 |
| Acinetobacter baumannii | M6557931 | Acinetobacter baumannii | T6960905 |
| Acinetobacter baumannii | M6570364 | Acinetobacter baumannii | W6892187 |
| Escherichia coli | YDC107 YD | Acinetobacter baumannii | X4370779 |
| Escherichia coli | YDC107 TBD | Acinetobacter baumannii | W6640817 |
| Escherichia coli | CA11 | Acinetobacter baumannii | T6781787 |
| Klebsiella pneumoniae | C2 | Acinetobacter baumannii | M6727536-1 |
| Klebsiella pneumoniae | D7 | Acinetobacter baumannii | X4324443 |
| Pseudomonas aeruginosa | PAO1 | Acinetobacter baumannii | S4825574 |
| Staphylococcus aureus | MRSA DOH 040 | Acinetobacter baumannii | S4823762 |
| Staphylococcus aureus | MRSA DOH 075 | Acinetobacter baumannii | W6910684 |
| Enterococcus | VRE 24670 | Acinetobacter baumannii | M6634593 |
| Enterococcus | VRE 26692 | Acinetobacter baumannii | S4892351 |
| Acinetobacter baumannii | 1A3 TBD | Acinetobacter baumannii | X4230972 |
| Acinetobacter baumannii | 1F8 TBD | Acinetobacter baumannii | M6594033 |
| Acinetobacter baumannii | M6547155 | Acinetobacter baumannii | S4699435 |
| Acinetobacter baumannii | F6295668 | Acinetobacter baumannii | W6711824 # 1 |
| Acinetobacter baumannii | T6705170 | Acinetobacter baumannii | W6711824 # 2 |
| Acinetobacter baumannii | F6391360 | Acinetobacter baumannii | X4206386 |
| Acinetobacter baumannii | S4684993 | Acinetobacter baumannii | F6316522 |
| Acinetobacter baumannii | S4686203 | Acinetobacter baumannii | F6388319 |
| Acinetobacter baumannii | M6654224 | Acinetobacter baumannii | W6720874 |
| Acinetobacter baumannii | W6693747 | Acinetobacter baumannii | F6504088 |
| Acinetobacter baumannii | W6922298 | Acinetobacter baumannii | |
| Acinetobacter baumannii | X4369531 | Acinetobacter baumannii | M7258938 |
| Acinetobacter baumannii | F6636182 | Acinetobacter baumannii | H7234039 |
| Acinetobacter baumannii | H6901665 | Acinetobacter baumannii | T7395013 |
| Acinetobacter baumannii | F6652813 | Acinetobacter baumannii | X4624820 |
| Acinetobacter baumannii | S4887595 | Acinetobacter baumannii | H7283745 |
| Acinetobacter baumannii | S4887596 | Acinetobacter baumannii | S5146483 |
| Acinetobacter baumannii | M7001534 | Acinetobacter baumannii | S5144766 |
| Acinetobacter baumannii | T7118427 | Acinetobacter baumannii | S5144767 |
| Acinetobacter baumannii | W7026767 | Acinetobacter baumannii | S5153022 |
| Acinetobacter baumannii | W7027329 | Acinetobacter baumannii | F7045731 |
| Acinetobacter baumannii | H7041121 | Acinetobacter baumannii | F7044161 |
| Acinetobacter baumannii | M7082089 | Acinetobacter baumannii | S5175684 |
| Acinetobacter baumannii | M7082123 | Acinetobacter baumannii | H7342951 |
| Acinetobacter baumannii | H7062449 | Acinetobacter baumannii | F7071176 |
| Acinetobacter baumannii | M7108041 | Acinetobacter baumannii | F7077571 |
| Acinetobacter baumannii | T7202985 | Acinetobacter baumannii | F7077571 |
| Acinetobacter baumannii | T7244103 | Acinetobacter baumannii | F7071329 |
| Acinetobacter baumannii | H7110321 | Acinetobacter baumannii | S5190099 |
| Acinetobacter baumannii | H7104234 | Acinetobacter baumannii | X4689437 |
| Acinetobacter baumannii | M7156070 | Acinetobacter baumannii | X4689555 |
| Acinetobacter baumannii | F6871068 | Acinetobacter baumannii | M7426099 |
| Acinetobacter baumannii | W7197258 | Acinetobacter baumannii | T7513368 |
| Acinetobacter baumannii | W7208924 | Acinetobacter baumannii | T7529070 |
| Acinetobacter baumannii | W7210606 | Acinetobacter baumannii | T7513462 |
| Acinetobacter baumannii | F6926143 | Acinetobacter baumannii | H7398342 |
| Acinetobacter baumannii | H7182108 | Acinetobacter baumannii | M7435333 |
| Acinetobacter baumannii | H6879046 | Acinetobacter baumannii | H7398342 |
| Acinetobacter baumannii | M6945349 | Acinetobacter baumannii | T7580580 |
| Acinetobacter baumannii | H6934427 | Acinetobacter baumannii | H7449015 |
| Acinetobacter baumannii | F6715105 | Acinetobacter baumannii | H7449650 |
| Acinetobacter baumannii | T7187783 | Acinetobacter baumannii | F7174333 |
| Acinetobacter baumannii | F6843354 | Acinetobacter baumannii | T7570091 |
| Acinetobacter baumannii | F6847922 | Acinetobacter baumannii | T7570089 |
| Acinetobacter baumannii | M7150740 | Acinetobacter baumannii | M7498119 |
| Acinetobacter baumannii | T7268129 | Acinetobacter baumannii | T7196382 |
| Acinetobacter baumannii | M7191688 | Acinetobacter baumannii | X4754725 |
| Acinetobacter baumannii | M7211623 | Acinetobacter baumannii | S5301575 |
| Acinetobacter baumannii | T7315372 | Acinetobacter baumannii | M7536545 |
| Acinetobacter baumannii | W6959400 | Acinetobacter baumannii | W7581338 |
| Acinetobacter baumannii | T7150606 | Acinetobacter baumannii | M7574228 |
| Acinetobacter baumannii | T7286950 | Acinetobacter baumannii | M7576584 |
| Acinetobacter baumannii | T7364864 | Acinetobacter baumannii | F7382345 |
| Acinetobacter baumannii | H7229162 | Acinetobacter baumannii | T7852665 |
| Acinetobacter baumannii | M7282089 | Acinetobacter baumannii | F7418055 |
| Acinetobacter baumannii | T7450967 | Acinetobacter baumannii | S5434534 |
| Acinetobacter baumannii | H7308283 | Acinetobacter baumannii | T7876307 |
| Acinetobacter baumannii | X4652199 | Acinetobacter baumannii | S5462569 |
| Acinetobacter baumannii | F7053276 | Acinetobacter baumannii | S5476955-1 |
| Acinetobacter baumannii | T7469180 | Acinetobacter baumannii | S5476955-2 |
| Acinetobacter baumannii | H7342303 | Acinetobacter baumannii | F7491014 |
| Acinetobacter baumannii | H7332154 # 1 | Acinetobacter baumannii | X4952534 |
| Acinetobacter baumannii | H7332154 # 2 | Acinetobacter baumannii | T7943002 |
| Acinetobacter baumannii | H7342303 # 2 | Acinetobacter baumannii | T8009201 |
| Acinetobacter baumannii | S5189078 | Acinetobacter baumannii | H7869473 |
| Acinetobacter baumannii | X4675691 | Acinetobacter baumannii | H7555054 |
| Acinetobacter baumannii | F7096051 | Acinetobacter baumannii | T7717740 |
| Acinetobacter baumannii | F7096452 | Acinetobacter baumannii | W7615302 |
| Acinetobacter baumannii | W7519025 | Acinetobacter baumannii | S5342757 |
| Acinetobacter baumannii | X4785831 | Acinetobacter baumannii | T7750534 |
| Acinetobacter baumannii | T7693515 | Acinetobacter baumannii | T7750534 |
| Acinetobacter baumannii | T7693524 | Acinetobacter baumannii | H7598358 |
| Acinetobacter baumannii | M7374618 # 1 | Acinetobacter baumannii | X4838376 |
| Acinetobacter baumannii | M7374618 # 2 | Acinetobacter baumannii | T7766780 |
| Acinetobacter baumannii | W7553904 | Acinetobacter baumannii | W7671817 |
| Acinetobacter baumannii | W7578283 | Acinetobacter baumannii | H7635547 |
| Acinetobacter baumannii | F6948309 | Acinetobacter baumannii | M7709402 |
| Acinetobacter baumannii | W7290980 | Acinetobacter baumannii | T7820160 |
| Acinetobacter baumannii | M7310854 | Acinetobacter baumannii | F7390009 |
| Acinetobacter baumannii | M7392235 | Acinetobacter baumannii | W7734369 |
| Acinetobacter baumannii | W7484162 | Acinetobacter baumannii | S5459386 |
| Acinetobacter baumannii | W7601810 | Acinetobacter baumannii | S5461928 |
| Acinetobacter baumannii | F7271515 | Acinetobacter baumannii | X4936260 |
| Acinetobacter baumannii | S5335610 | Acinetobacter baumannii | M7316381 |
| Acinetobacter baumannii | H7579564 | Acinetobacter baumannii | S5495509 |
| Acinetobacter baumannii | X4831716 | Acinetobacter baumannii | M7838140 |
| Acinetobacter baumannii | F7298304 | Acinetobacter baumannii | W7854006 |
| Acinetobacter baumannii | H7610715 | Acinetobacter baumannii | T7973820 |
| Acinetobacter baumannii | T7772783 | Acinetobacter baumannii | W7891295 |
| Acinetobacter baumannii | M7712644 | Acinetobacter baumannii | H7842805 |
| Acinetobacter baumannii | M7712658 | Acinetobacter baumannii | M7877907 |
| Acinetobacter baumannii | T7822340 | Acinetobacter baumannii | T8000438 |
| Acinetobacter baumannii | H7685900 | Acinetobacter baumannii | H7872553 |
| Acinetobacter baumannii | H7693024 | Acinetobacter baumannii | M7896154 |
| Acinetobacter baumannii | M7914463 | Streptococcus mitis | POS 4489 |
| Acinetobacter baumannii | W7930548 | Streptococcus mitis | POS 5586 |
| Acinetobacter baumannii | F7316875 | Streptococcus mutans | POS 5593 |
| Acinetobacter baumannii | T7796673 | Streptococcus mutans | POS 1260 |
| Acinetobacter baumannii | T7850239 | Streptococcus pneumoniae | POS 10164 |
| Acinetobacter baumannii | W7763340 | Streptococcus pneumoniae | POS 6892 |
| Acinetobacter baumannii | M7837667 | Staphylococcus haemolyticus | POS 8764 |
| Acinetobacter baumannii | F7518628 | Staphylococcus lugdunensis | POS 10768 |
| Candida albicans | YST 1032 | Staphylococcus lugdunensis | POS 8659 |
| Candida albicans | YST 1369 | Streptococcus mitis | POS 4489 |
| Candida albicans | YST 1862 | Streptococcus mitis | POS 5586 |
| Escherichia coli | ENF 18187 | Streptococcus mutans | POS 5593 |
| Enterobacter aerogenes | ENF 10856 | Streptococcus mutans | POS 1260 |
| Enterobacter aerogenes | ENF 11218 | Streptococcus pneumoniae | POS 10164 |
| Enterobacter aerogenes | ENF 11237 | Streptococcus pneumoniae | POS 6892 |
| Klebsiella oxytoca | ENF 3950 | Streptococcus pneumoniae | POS 6289 |
| Klebsiella oxytoca | ENF 4321 | Streptococcus sanguinis | POS 5589 |
| Klebsiella oxytoca | ENF 11686 | Streptococcus sanguinis | POS 4696 |
| Klebsiella pneumoniae | ENF 16491 | Strains identified in blood bottles | |
| Klebsiella pneumoniae | ENF 18027 | Escherichia coli | W3110 |
| Pseudomonas aeruginosa | ENF 17948 | Pseudomonas aeruginosa | PAO1 |
| Staphylococcus epidermidis | POS 6544 | Acinetobacter baumannii | 1I7 |
| Staphylococcus epidermidis | POS 10235 | Klebsiella pneumoniae | B6 |
| Staphylococcus haemolyticus | POS 10866 | Staphylococcus aureus | M2 |
| Staphylococcus haemolyticus | POS 8764 | Staphylococcus aureus | NRS123 |
| Staphylococcus lugdunensis | POS 10768 | | |
| Staphylococcus lugdunensis | POS 8659 | | |

The following references are cited herein:
1. El Hamidi et al., J. Lipid Res., 2005, 46:1773-1778.
2. Tirsoaga et al. J Lipid Res. 2007, 48(11):2419-27.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention.

What is claimed is:

1. A method for rapidly identifying a microbe, comprising:
obtaining a biological sample from a subject;
determining, via spectrometry, a molecular mass profile of microbial lipids; and
comparing the molecular mass profile of the lipids from the microbe with a molecular mass profile of lipids from a known microbe wherein an identical profile indicates the identity of the microbe in the biological sample.

2. The method of claim 1, further comprising:
isolating the microbe from the biological sample.

3. The method of claim 1, wherein the determining step comprises:
extracting lipids from the microbe prior to the spectrometry; or
performing spectrometry on microbial cells.

4. The method of claim 1, wherein the spectrometry is mass spectrometry, tandem mass spectrometry or ion mobility spectrometry.

5. The method of claim 1, wherein the lipid is lipid A, Lipoteichoic Acid, a glycolipid, or cardiolipin.

6. The method of claim 1, wherein the microbe is a pathogen, a non-pathogen or a commensal bacterium.

7. The method of claim 6, wherein the pathogen is selected from the group consisting of Acinetobacter; Actinomyces, Arthrobacter, Burkholderia, Bacillus, Bacteroides, Bordetella, Borrelia, Brevundimonas, Brucella, Candida, Clostridium, Corynebacterium, Campylobacter, Deinococcus, Escherichia, Enterobacter, Enterococcus, Erwinia, Eubacterium, Exiguobacterium, Flavobacterium, Francisella, Gluconobacter, Helicobacter, Intrasporangium, Janthinobacterium, Klebsiella, Kingella, Legionella, Leptospira, Mycobacterium, Moraxella, Micrococcus, Neisseria, Oscillospira, Proteus, Pseudomonas, Providencia, Paenibacillus, Rickettsia, Rhodococcus, Roseomonas, Rothia, Salmonella, Serratia, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

8. The method of claim 1, wherein the microbe is a Gram-negative bacterium and the lipid is lipid A, a glycolipid or cardiolipin.

9. The method of claim 1, wherein the microbe is a Gram-positive bacterium and the lipid is Lipoteichoic Acid, a glycolipid or cardiolipin.

10. The method of claim 1, wherein the microbe is a fungus and the lipid is a glycolipid or cardiolipin.

11. The method of claim 1, wherein the biological sample is blood, urine, stool, serum, wound effluent or bronchoalveolar lavage fluid.

12. A method for rapidly identifying a pathogenic bacterium in a blood sample comprising:
obtaining the blood sample from a subject;
extracting lipids from the bacterial pathogen at zero passage;
determining, via spectrometry, a molecular mass profile of the extracted lipids; and
comparing the molecular mass profile of the extracted lipids with a molecular mass profile of lipids from a known pathogenic bacterium wherein an identical profile indicates the identity of the pathogenic bacteria.

13. The method of claim 12, further comprising:
isolating the pathogenic bacterium from the blood sample.

14. The method of claim 12, wherein the spectrometry is mass spectrometry, tandem mass spectrometry or ion mobility spectrometry.

15. The method of claim 12, wherein the pathogenic bacterium is selected from the group consisting of Acinetobacter, Actinomyces, Arthrobacter, Burkholderia, Bacillus, Bacteroides, Bordetella, Borrelia, Brevundimonas, Brucella, Clostridium, Corynebacterium, Campylobacter, Deinococcus, Escherichia, Enterobacter, Enterococcus, Erwinia, Eubacterium, Exiguobacterium, Flavobacterium, Francisella, Gluconobacter, Helicobacter, Intrasporangium, Janthinobacterium, Klebsiella, Kingella, Legionella, Leptospira, Mycobacterium, Moraxella, Micrococcus, Neisseria, Oscillospira, Proteus, Pseudomonas, Providencia, Paenibacillus, Rickettsia, Rhodococcus, Roseomonas, Rothia, Salmonella, Serratia, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

16. The method of claim 12, wherein the pathogenic bacterium is a Gram-negative bacterium and the lipid is lipid A, a glycolipid or cardiolipin or the pathogenic bacterium is Gram-positive bacterium and the lipid is Lipoteichoic Acid, a glycolipid or cardiolipin.

17. A method for identifying one or more antimicrobial drugs effective to treat a microbial strain in a subject in need of such treatment, comprising:
obtaining a biological sample from the subject;
extracting lipids from microbes in the sample,
performing a spectrographic analysis of the extracted lipids to obtain a molecular mass profile thereof;
comparing the extracted lipids profile with a library of known lipid profiles of strains of pathogenic microbes to identify the strain of pathogenic microbe in the biological sample; and
identifying one or more antimicrobial drugs effective to treat the identified microbial strain.

18. The method of claim 17, wherein the performing step comprises analysis via mass spectrometry, tandem mass spectrometry or ion mobility spectrometry.

19. The method of claim 18, wherein the spectrographic analysis of lipids differentiates among pathogenic microbes at the genus, species, sub-species or strain level.

20. The method of claim 17, wherein the pathogenic microbe is a Gram-negative bacterium, a Gram-positive bacterium or a fungus.

21. The method of claim 20, wherein the Gram-negative bacterial lipid is lipid A, a glycolipid or cardiolipin, the Gram-positive bacterial lipid is Lipoteichoic Acid, a glycolipid or cardiolipin and the fungal lipid is a glycolipid precursor of one or both of lipid A or Lipoteichoic Acid or a cardiolipin.

22. The method of claim 17, wherein the biological sample is blood, urine, stool, serum, wound effluent or bronchoalveolar lavage fluid.
